(19) United States
(12) Patent Application Publication
(10) Pub. No.: US 2018/0184894 A1
(43) Pub. Date: Jul. 5, 2018

(54) ULTRA-WIDE FIELD OF VIEW OPTICAL COHERENCE TOMOGRAPHY IMAGING SYSTEM

(71) Applicant: VISUNEX MEDICAL SYSTEMS CO. LTD., Grand Cayman (KY)

(72) Inventor: Wei Su, Sunnyvale, CA (US)

(21) Appl. No.: 15/847,843

(22) Filed: Dec. 19, 2017

Related U.S. Application Data
(60) Provisional application No. 62/436,201, filed on Dec. 19, 2016.

(51) Int. Cl. A61B 3/10; G01B 9/02; A61B 3/15

(52) U.S. Cl. CPC A61B 3/102; G01B 9/02091; A61B 5/0066; G01B 9/02041; A61B 3/15

(57) ABSTRACT

Various embodiments of the present disclosure describe an ultra-wide field of view (FOV) optical coherence tomography (OCT) imaging system. The ultra-wide FOV OCT imaging system can include an imaging probe, a console and a cable. The imaging probe can include an optical widow, a first imaging module and a second imaging module. The first imaging module is configured to form a first image of the eye. The second imaging module is configured to form a second image of the eye. The second imaging module can include a scanning mirror configured to receive a sample arm portion of a second light beam from a second light source and scan the sample arm portion. The console can include the second light source, an interferometer, and a processor. The cable is coupled between the console and the imaging probe and includes a first fiber, a second fiber and a third fiber.

### CROSS REFERENCE TO RELATED APPLICATIONS

This application claims the benefit of U.S. Provisional Application No. 62/436,201, titled: “OPTICAL COHERENCE TOMOGRAPHY IMAGING SYSTEM WITH A REMOTE IMAGING PROBE”, filed on Dec. 19, 2016, which is hereby incorporated by reference herein in its entirety.

### INCORPORATION BY REFERENCE

The following U.S. patent applications are hereby incorporated herein by reference in their entireties to the same extent as if each individual publication or patent application was specifically and individually indicated to be incorporated by reference: U.S. patent application Ser. No. 15/186,402, titled: “WIDE FIELD OF VIEW OPTICAL COHERENCE TOMOGRAPHY IMAGING SYSTEM” and filed Jun. 17, 2016, U.S. Pat. No. 9,155,466, titled “EYE IMAGING APPARATUS WITH A WIDE FIELD OF VIEW AND RELATED METHODS” and filed on Feb. 4, 2015, U.S. patent application Ser. No. 14/220,005, titled “EYE IMAGING APPARATUS AND SYSTEMS”, filed on Mar. 19, 2014, U.S. patent application Ser. No. 14/312,590, titled “MECHANICAL FEATURES OF AN EYE IMAGING APPARATUS”, filed on Jun. 23, 2014, U.S. patent application Ser. No. 15/007,101, titled: “A DISPOSABLE CAP FOR AN EYE IMAGING APPARATUS AND RELATED METHODS” and filed on Jan. 26, 2016, and U.S. Patent Application No. 62/141,209, titled “A WIRELESS IMAGING APPARATUS AND RELATED METHODS”, filed on Mar. 31, 2015.

All publications and patent applications mentioned in this specification are herein incorporated by reference in their entirety to the same extent as if each individual publication or patent application was specifically and individually indicated to be incorporated by reference.

### BACKGROUND

Various embodiments of the disclosure relate generally to an Optical Coherence Tomography (OCT) imaging system, particularly, an ultra-wide field Optical Coherence Tomography (OCT) imaging system with a remote imaging probe.

Eyes are among the most valued human organs that play indispensable roles in human life. Likewise, eye diseases and vision loss in general are serious problems. Moreover, eye diseases and vision problems among children, especially new-born babies, can have severe and far-reaching implications. For infants and small children, the visual centers in the brain are not fully mature. For the visual centers in the brain to develop properly, proper input from both eyes is desirable. Therefore, good vision can be an important factor in the proper physical development and educational progress. Undetected eye problems in infants and others may result in irreversible loss of vision. For example, retinopathy of prematurity in prematurely born infants is one of the leading causes of childhood blindness in the world. Early detection and diagnosis provide the best opportunity for treatment and prevention of vision loss. Since retinal and optic nerve problems are among the leading causes in vision loss, an eye imaging system capable of imaging a posterior segment of the eye can be particularly useful. Moreover, an eye imaging system with a wide field of view can offer the benefit of enabling evaluation of pathologies located on the periphery of the retina.

Optical coherence tomography (OCT) imaging has revolutionized eye examination since its inception. The OCT imaging systems have achieved widespread adoption in clinical ophthalmology because of the ability to provide the structure information of the eye. In general, the OCT imaging systems typically use near-infrared light to non-invasively create volumetric images of an eye, such as cross-sectional views and three dimensional views of an eye. For example, the OCT imaging systems can provide visualization of retinal tissue in cross-sectional views and three dimensional views.

Conventional OCT imaging systems can only provide a field of view (FOV) of about 20°×20°. This FOV is not sufficient to observe the periphery of the retina. The periphery of the retina offers a unique perspective towards the assessment and monitoring of certain ocular diseases, including diabetic retinopathy, retinal vein occlusion, and choroid masses. It is important to detect architectural changes caused by peripheral retinal pathology to aid in the detection and diagnosis of various eye diseases. For example, the importance of the retinal periphery is well known for diseases such as diabetic retinopathy or vascular occlusions. For another example, even for diseases thought to be exclusively diseases of the fovea such as age-related macular degeneration, an interdependency of the retinal periphery to macular changes is recognized. However, the FOV of the conventional OCT imaging systems are limited due to limited axial depth range of the OCT imaging system, field curvature of the retina, motion artifacts, backscattering of the retina, optical system aberrations, and other problems. The technique of mosaicking has been used to achieve a larger FOV. However, a major drawback of mosaicking is the increased measurement time for a whole volume, including the time to realign the patient. Post-processing also becomes more complex with possible registration errors.

In addition, the conventional OCT systems suffer from high noise and instability of the performance because of the inevitable relative movement of the OCT imaging system and the eye of the patient. This problem is more severe for a large FOV OCT imaging system.

Furthermore, most conventional OCT imaging systems have been non-contact type tabletop systems. In general, the conventional OCT imaging systems typically have a chin rest for a patient and a mechanism for aligning the patient with the OCT imaging system. This system typically requires a mobile, upright, and cooperative patient in order to obtain stable OCT images. However, the conventional non-contact OCT imaging systems require the cooperation of the patients, which is impractical for small children and adult patients who are bedridden or non-cooperative. The conventional non-contact OCT systems will not be able to obtain OCT images of animal eyes as well.

Contact-type OCT imaging probes have been proposed recently. For example, a portable OCT imaging probe with a contact lens is proposed in U.S. Pat. No. 9,173,563. Portable probes may be useful in retinal imaging for children, adults that are not cooperative or are bedridden, animals, etc. The contact probe makes physical contact between the probe and the patient; alignment may therefore be relatively simple. However, prior contact-type OCT imaging probes, including the optical design in the above '563 patent, suffer from several problems. For example, the achievable field of view (“FOV”) in prior contact-type OCT imaging probes is limited because of strong reflected light from the cornea resulted from the optical design. In another example, high noise in the OCT imaging system due to the unavoidable motions of the fibers during operation significantly decrease the stability and result in low quality of the OCT images, thus not being practical in clinical applications.

As the foregoing illustrated, there is need to develop an ultra-wide field of view OCT imaging system to observe the periphery of the retina. Furthermore, there is a need to develop an OCT imaging system with an imaging probe with high stability and low noise for clinical applications, which is suitable for infants, small children, bedridden patients, animals, etc.

### SUMMARY OF THE DISCLOSURE

The present disclosure relates to an ultra-wide field Optical Coherence Tomography (OCT) imaging system with a remote imaging probe. Various embodiments of the present disclosure describe an ultra-wide field of view (FOV) optical coherence tomography (OCT) imaging system. The ultra-wide FOV OCT imaging system can include an imaging probe. The imaging probe can include an optical widow configured to be in contact with a cornea of an eye. The imaging probe can include a first imaging module and a second imaging module. The first imaging module is configured to form a first image of the eye. For example, the first imaging module can be a two dimensional (2D) color imaging module. The first imaging module can include a first light source and a light conditioning element, where the first light source is configured to provide a first light beam and the light conditioning element is configured to direct the first light beam through the optical window to the eye. The second imaging module is configured to form a second image of the eye. For example, the second imaging module can be an OCT imaging module to provide a volumetric image of the eye, such as a cross-sectional view, an en face view, or a three-dimensional view of the eye. The second imaging module can include a scanning mirror configured to receive a sample arm portion of a second light beam from a second light source and scan the sample arm portion. The second light source can be disposed on a console, or in the imaging probe. The second light beam of the second light source can include the sample arm portion and a reference arm portion. The ultra-wide FOV OCT imaging system can further include a beam splitter, for example, a beam splitting dichroic mirror, configured to transmit the first light beam and to reflect the sample arm portion of the second light beam.

In some embodiments, the ultra-wide FOV OCT imaging system can further include a console and a cable coupled between the console and the imaging probe. The console can include the second light source, an interferometer, and a processor. The interferometer is configured to receive the reflected light from the second imaging module and to generate data of the interference between the sample arm portion and the reference arm portion of the second beam. The processor is configured to process the data from the interferometer and to generate the second image, which is the OCT image. The cable can include a first fiber and a second fiber, where the first fiber is configured to transmit the sample arm of the second light beam to the second imaging module in the imaging probe and to transmit reflected light of the sample arm from the second imaging module to the interferometer in the console; where the second fiber is configured to transmit a reference arm of the second light beam from the second light source to the second imaging module in the imaging module. In some embodiments, the cable further includes a third fiber, the third fiber being configured to transmit reflected light of the reference arm portion from the second imaging module to the interferometer. In some embodiments, the first fiber, the second fiber and the third fiber are closely fixed inside the cable such that external motion effects cause same changes in polarization direction and optical path lengths for the first fiber, the second fiber and the third fiber. In some embodiments, the second fiber is configured to transmit reflected light of the reference arm portion from the second imaging module to the interferometer.

In some embodiments, the ultra-wide FOV OCT imaging system can include an optical path difference (OPD) compensator disposed in an optical path of the sample arm portion, the OPD compensator including a center and a peripheral region, wherein a first optical path along the center is shorter than a second optical path along the peripheral region. In some embodiments, the OPD compensator is disposed closely to a secondary image plane of the second imaging module within 5 mm. In some embodiments, the optical path difference between the first optical path and the second optical path is between 0.5 mm to 3 mm.

In some embodiments, the ultra-wide FOV OCT imaging system can include a dispersion compensation module. The dispersion compensation module can be disposed in an optical path of the reference arm portion and configured to perform dispersion compensation of the OCT imaging system. In some embodiments, the second imaging module, the OCT imaging module, can include a plurality of optical lenses, where the plurality of optical lenses are configured for full FOV dispersion compensation. The plurality of optical lenses can include a plurality of optical materials. The plurality of optical materials can be selected such that a difference between a first total dispersion of a first optical path length of the sample arm portion in the center and a second total dispersion of a second optical path length of the sample arm portion along the peripheral region of a full FOV is reduced, thereby an axial resolution of a second image is improved. The total dispersion is the dispersion between two extreme wavelengths, a longest wavelength and a shortest wavelength in a wavelength range of the second light source. In some embodiments, the plurality of optical lenses are configured to reduce an optical path difference (OPD) between the longest wavelength and the shortest wavelength in the wavelength range of the second light source to be a constant for the full FOV of the OCT imaging system by selecting the plurality of optical materials. In some embodiments, the dispersion compensation module is configured to compensate a residual OPD for the full FOV of the OCT imaging system. The residual OPD is the remaining OPD between the longest wavelength and the shortest wavelength in the wavelength range of the second light source for the full FOV after OPD being reduced or compensated by the plurality of optical lenses through selecting the plurality of optical materials.

In some embodiments, the ultra-wide FOV OCT imaging system can include a two-channel optical path difference (OPD) compensation unit disposed in an optical path of the reference arm portion and including a first optical channel, a second optical channel, and an optical switching element, wherein a first optical path of the first optical channel is shorter than a second optical path of the second optical channel, wherein the optical switching element is configured to switch the reference arm portion between the first optical channel and the second optical channel such that an axial optical depth of the OCT imaging system is doubled, thereby a field of view of the OCT imaging system is extended.

Disclose herein is an ultra-wide field of view (FOV) optical coherence tomography (OCT) imaging system including an optical circulator. The OCT system can include an optical window, a first imaging module and a second imaging module. The optical window is disposed at a distal end of an imaging probe and configured to be in contact with a cornea of an eye. The first imaging module is disposed inside the imaging probe and configured to form a first image of the eye. The first imaging module can include a first light source and a light conditioning element. The first light source is configured to provide a first light beam. The light conditioning element is configured to direct the first light beam through the optical window to the eye. The second imaging module is disposed inside the imaging probe and configured to receive a second light beam from a second light source. The second light source is configured to provide the second light beam. The second light beam includes a sample arm portion and a reference arm portion. The OCT system can further include a beam splitter disposed inside the imaging probe and configured to transmit the first light beam and to reflect the sample arm portion of the second light beam. The OCT system can further include the optical circulator disposed in an optical path of a light beam portion. The light beam portion includes at least one of the sample arm portion or the reference arm portion. The optical circulator includes a first port, a second port, and a third port. The first port is configured to receive the light beam portion and to transmit the light beam portion to the second port. The second port is configured to transmit the light beam portion out, for example, to one of a first fiber or a second fiber, and to receive retuned light of the light beam portion and to transmit the retuned light of the light beam portion to the third port.

Advantageously, the OCT imaging system can have an ultra-wide field of view (FOV). Due to the contact nature of the OCT imaging module, a center of eye (more precisely, a center of the radius of curvature of the retina when being considered to be in a spherical shape) is used as a point to measure the FOV. In some embodiments, the FOV is 120 degrees×120 degrees and up to 180 degrees×180 degrees in a single volume acquisition. In some embodiments, the FOV is 130 degrees×130 degrees and up to 180 degrees×180 degrees in a single volume acquisition. In terms of a scanning line on the retina, in some embodiments, the FOV of the OCT imaging system is about at least 20 mm×20 mm for new born infants, 24 mm×24 mm for children, and 28-30 mm×28-30 mm for adults, in a single volume acquisition. Because the periphery of the retina offers a unique perspective towards the assessment and monitoring of certain ocular diseases, the ultra-wide field of view of the OCT imaging system is valuable in detecting architectural changes caused by peripheral retinal pathology for the detection and diagnosis of various eye diseases.

In addition, the ultra-wide FOV OCT imaging system with an imaging probe is also advantageous to provide more stable OCT images and increase the image quality significantly. In some embodiments, the OCT imaging system includes a triple fiber configuration and the related optical design for the OCT interferometer. Because the three fibers are closely fixed inside one single long cable, the external effects, such as motion effect from stretching, bending or twisting of the fibers, would change the polarization direction as well as optical path of light beam transmitting in the fibers in same fashion and same amount. Thus, such motion induced optical effect can be cancelled out or minimized by the OCT interferometer. Therefore, the resulted OCT images are more stable with significantly increased image quality.

Furthermore, Moreover, the OCT imaging system is advantageous for infants, small children, bedridden patients, and animals because of easy alignment. The OCT imaging system can minimize the lateral alignment requirement between the eye of the user and the optical system of the OCT imaging system. Because the shape of the contact optical window is designed to fit closely with the shape of the cornea, it is easier to align the contact optical window with the cornea. The contact optical window can also reduce the free motion of the eye ball too. The OCT imaging system can provide high quality images for a group of users whose OCT images are difficult to be obtained by the conventional OCT imaging systems.

### BRIEF DESCRIPTION OF THE DRAWINGS

FIG. 1A is a side view of an ultra-wide field of view (FOV) optical coherence tomography (OCT) imaging system with an imaging probe according to various embodiments of the disclosure.

FIG. 1B is a side view of an ultra-wide FOV optical coherence tomography (OCT) imaging system with an imaging probe according to some other embodiments of the disclosure.

FIG. 1C is a photo of a prototype of the ultra-wide FOV OCT imaging system in FIG. 1A.

FIG. 2A is a schematic of an example optical design of an ultra-wide FOV OCT imaging system with an imaging probe, where a posterior segment of an eye is imaged, according to one embodiment of the disclosure.

FIG. 2B illustrates one embodiment of a reflection module in the OCT imaging system in FIG. 2A.

FIG. 2C illustrates an optical fiber cable in the OCT imaging system in FIG. 2A, according to one embodiment of the disclosure.

FIG. 2D illustrates another optical fiber cable in the OCT imaging system in FIG. 2A, according to another embodiment of the disclosure.

FIG. 3A illustrates an OCT image of a posterior segment of the eye, where a folding in phenomena is exhibited at a left-upper corner.

FIG. 3B is a schematic of an example optical design of an ultra-wide FOV OCT imaging system with an optical path difference (OPD) compensator according to some embodiments of the disclosure.

FIG. 4A illustrates a block diagram of an ultra-wide FOV OCT imaging system with a dispersion compensation module according to one embodiment of the disclosure.

FIG. 4B illustrates a reflection module of the ultra-wide FOV OCT imaging system in FIG. 4A.

FIG. 4C illustrates a fiber cable of the ultra-wide FOV OCT imaging system in FIG. 4A.

FIG. 4D illustrates a dispersion plot of one optical material FPL51, which shows different index of refraction at different wavelengths for FPL51.

FIG. 4E illustrates a dispersion plot of another optical material TIH53, which shows different index of refraction at different wavelengths for TIH53.

FIG. 4F illustrates a dispersion plot of yet another optical material Quartz, which shows different index of refraction at different wavelengths for Quartz.

FIG. 5 is a block diagram of an OCT imaging system including a circulator in a reference arm portion according to one embodiment of the disclosure.

FIG. 6 is a block diagram of an OCT imaging system including a first circulator in a reference arm portion and a second circulator in a sample arm portion according to another embodiment of the disclosure.

FIG. 7 is a block diagram of an OCT imaging system including a circulator in a sample arm portion according to another embodiment of the disclosure.

FIG. 8A is a block diagram of an OCT imaging system including a two-channel OPD compensation unit according to one embodiment of the disclosure.

FIG. 8B illustrates an optical path difference between an anterior segment and a posterior segment of the eye.

FIG. 8C illustrates details of the two-channel OPD compensation unit in FIG. 8A.

FIG. 8D illustrates a two-channel OPD compensation unit according to another embodiment of the disclosure.

FIG. 8E illustrates a two-channel OPD compensation unit according to yet another embodiment of the disclosure.

FIG. 8F illustrates an OCT image of a posterior segment of the eye, where a folding in phenomena is solved by optically unfolding.

### DETAILED DESCRIPTION

The present disclosure now will be described in detail with reference to the accompanying figures. This disclosure may be embodied in many different forms and should not be construed as limited to the exemplary embodiments discussed herein.

Various embodiments of the present disclosure describe an ultra-wide field of view (FOV) optical coherence tomography (OCT) imaging system. The ultra-wide FOV OCT imaging system can include an imaging probe. The imaging probe can include an optical widow configured to be in contact with a cornea of an eye. The imaging probe can include a first imaging module and a second imaging module. The first imaging module is configured to form a first image of the eye. For example, the first imaging module can be a two dimensional (2D) color imaging module. The first imaging module can include a first light source and a light conditioning element, where the first light source is configured to provide a first light beam and the light conditioning element is configured to direct the first light beam through the optical window to the eye. The second imaging module is configured to form a second image of the eye. For example, the second imaging module can be an OCT imaging module to provide a volumetric image of the eye, such as a cross-sectional view, an en face view, or a three-dimensional view of the eye. The second imaging module can include a scanning mirror configured to receive a sample arm portion of a second light beam from a second light source and scan the sample arm portion. The second light source can be disposed on a console, or in the imaging probe. The second light beam of the second light source can include the sample arm portion and a reference arm portion. The ultra-wide FOV OCT imaging system can further include a beam splitter configured to transmit the first light beam and to reflect the sample arm portion of the second light beam.

In some embodiments, the ultra-wide FOV OCT imaging system can include an optical path difference (OPD) compensator disposed in an optical path of the sample arm portion, the OPD compensator including a center and a peripheral region, wherein a first optical path along the center is shorter than a second optical path along the peripheral region.

In some embodiments, the ultra-wide FOV OCT imaging system can further include a console and a cable coupled between the console and the imaging probe. The console can include the second light source, an interferometer, and a processor. The interferometer is configured to receive the reflected light from the second imaging module and to generate data of the interference between the sample arm portion and the reference arm portion of the second beam. The processor is configured to process the data from the interferometer and to generate the second image, which is the OCT image. The cable can include a first fiber and a second fiber, where the first fiber is configured to transmit the sample arm of the second light beam to the second imaging module in the imaging probe and to transmit reflected light of the sample arm from the second imaging module to the interferometer in the console; where the second fiber is configured to transmit a reference arm of the second light beam from the second light source to the second imaging module in the imaging module. In some embodiments, the second fiber is further configured to transmit reflected light of the reference arm from the second imaging module to the interferometer in the console. In some embodiments, the cable further includes a third fiber, the third fiber being configured to transmit reflected light of the reference arm portion from the second imaging module to the interferometer. In some embodiments, the first fiber, the second fiber and the third fiber are closely fixed inside the cable such that external motion effects cause same changes in polarization direction and optical path lengths for the first fiber, the second fiber and the third fiber.

The imaging probe can comprise a probe housing with a distal end, where the optical window is disposed at the distal end. The first imaging module can comprise a first light source disposed inside the housing, where the first light source can have a first wavelength range. The optical window can have a concave front surface with a radius of curvature of from about 3 mm to about 18 mm. The optical window can be in contact of the cornea directly, or indirectly through a disposable cap. The index matching gel can be applied between the optical window and the cornea. The optical window is configured to match a radius of curvature of the cornea of the eye. The first imaging module can comprise the light conditioning element having multiple segments and positioned behind the peripheral portion of the optical window, the light conditioning element configured to directionally control a first light beam of the first light source to illuminate the eye through the first illumination path. The first imaging module can comprise a first focusing lens to adjust a first focus of the first imaging module, and an image sensor configured to receive a first image of the eye through a first imaging path. The first imaging path is separated from the first illumination path. The first imaging path does not overlap with the first illumination path. The light conditioning element is configured to direct substantially all the light exiting the light conditioning element outside an entrance pupil of the first imaging system. Further details of the first imaging module are disclosed in U.S. Pat. No. 9,155,466, which is herein incorporated by reference in its entirety.

The imaging probe can comprise a second imaging module, which is an OCT imaging module. The second imaging module can have a second illumination path and a second imaging path. The second imaging module can comprise a scanning MEMS mirror configured to scan a first portion of a second light beam from a second light source. The second light source can be an OCT light source, such as a broadband light source. In some embodiments, the second light source is a swept source laser, which can provide a fast scanning speed. The scanning MEMS mirror is disposed outside the first illumination path and the first imaging path. The OCT imaging system can comprise a beam splitting dichroic mirror disposed in the first imaging path and configured to transmit the first light beam and reflect the sample arm portion of the second light beam, and a second focusing lens to adjust a second focus of the second imaging module. Some details of the second imaging module are disclosed in U.S. patent application Ser. No. 15/186,402, which is herein incorporated by reference in its entirety.

The OCT imaging module can have an ultra-wide field of view (FOV). Due to the contact nature of the OCT imaging module, a center of eye (more precisely, a center of the radius of curvature of the retina when being considered to be in a spherical shape) is used as a point to measure the FOV. The FOV of the OCT imaging system, measured from the center of the eye, may in certain embodiments be at least 120 degrees×120 degrees and up to 180 degree×180 degrees in a single volume acquisition. In some embodiments, the FOV is at least 130 degrees×130 degrees but no more than 180 degrees×180 degrees in a single volume acquisition. In terms of a scanning line on the retina, in some embodiments, the FOV of the OCT imaging system is about at least 20 mm×20 mm for new born infants, 24 mm×24 mm for children, and 28-30 mm×28-30 mm for adults, in a single volume acquisition.

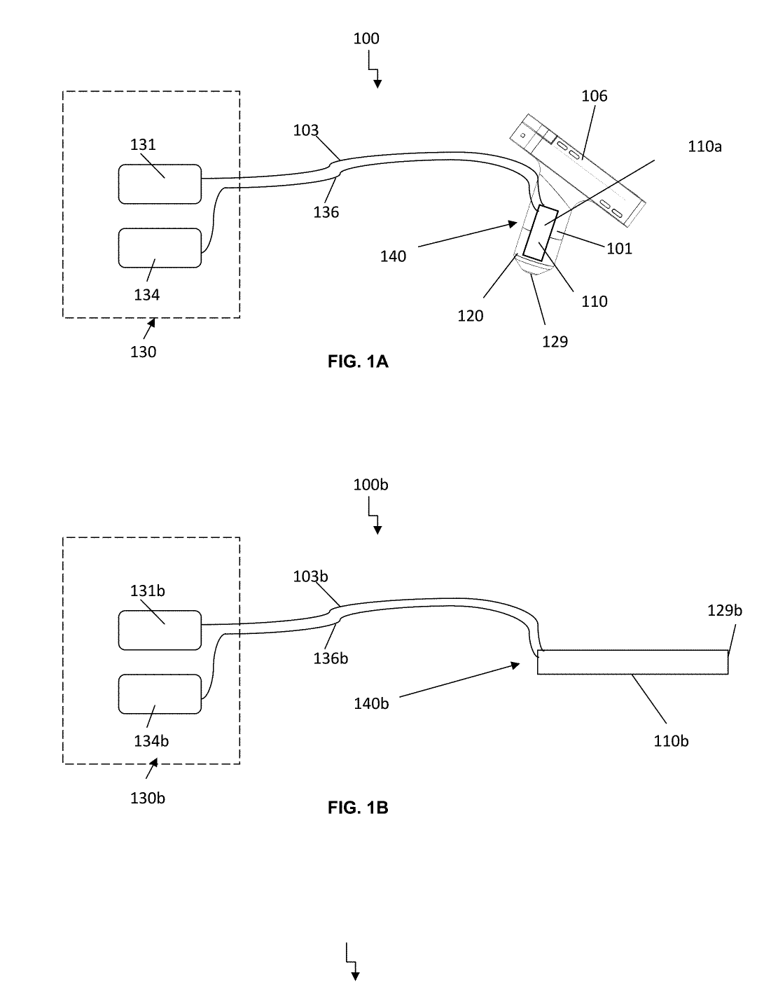

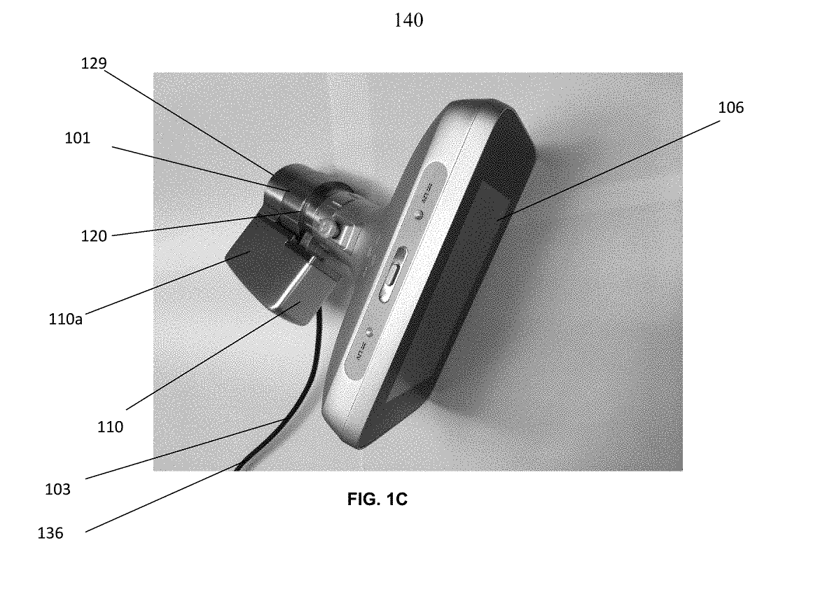

FIG. 1A is a side view of an optical coherence tomography (OCT) imaging system 100 having an imaging probe 140 and a console 130, according to some embodiments of the disclosure. FIG. 1C is a photo of a prototype of the ultra-wide FOV OCT imaging system in FIG. 1A. Referring to FIG. 1A and FIG. 1C, the handheld imaging probe 140 can comprise a probe housing with a distal end 129, a first imaging module 120 (for example, a color imaging module) and a second imaging module 110, which is an OCT imaging module 110. The OCT imaging system 100 can have an ultra-wide field of view. In general, the console 130 can comprise an OCT engine 131 and a scanning mirror controller 134. The OCT engine 131 can comprise an interferometer, a second light source (for example, an OCT light source with wavelength from 800 nm to 1100 nm), a light detector, a data acquisition system, a power supply, a processor, and a display. The second light source is an OCT light source. The OCT engine 131 can be Time-domain OCT engine (TD-OCT), Fourier-domain OCT engine (FD-OCT), or Swept source Fourier-domain OCT engine (SS-FD-OCT).

The handheld imaging probe 140 can be compact, light-weight and portable. The imaging probe 140 can comprise the first imaging module (for example, a color imaging module) 120 and the second imaging module 110, which is the OCT imaging module 110. In general, the imaging probe 140 houses not only the OCT imaging module, but also comes with other imaging modalities, for example, color imaging, fluorescein angiography imaging, auto-fluorescence imaging, etc. The imaging probe 140 can perform real-time OCT imaging with other imaging modality at same time, for example, the optical color imaging. In some embodiments, the optical color imaging module can be configured to provide registry of imaging locations for the OCT sectional views, which will be discussed in details below.

As also shown in FIG. 1A, an optical fiber 103 can be configured to transmit light from the second light source, which is an OCT light source, in the OCT engine 131 to the imaging probe 140. The OCT engine 131 can be linked, optically with the OCT imaging module 110 in the imaging probe 140 by the optical fiber 103, which supplies the light from the OCT light source to the OCT Imaging Module 110 and at same time, receive reflected light of the eye from the OCT Imaging Module 110 for further analysis. The OCT Imaging Module 110 scans the light beam from the OCT light source, by a scanning mirror (rotating one dimensionally or two dimensionally) disposed inside the handheld imaging probe 140, to the different parts of the eye (a posterior segment, for example, a retina, of the eye in the posterior imaging mode and an anterior segment of the eye in the anterior imaging mode) and receives the light reflected, or scattered, from the eye. For example, the scanning mirror can be a scanning MEMS mirror in some embodiments. An electrical cable 136 can be included as a date link 136 to connect the console 130 and the handheld imaging probe 140.

The handheld imaging probe 140 may be compact and in various embodiments, the imaging probe 140 may have a size less than 250 mm along the longest dimension thereof. For example, in some embodiments the imaging probe 140 may be between 250 mm and 200 mm, 150 mm, or 100 mm along the longest dimension. In some embodiments, the imaging probe 140 may weigh less than 3 kg or 6 lbs. For example, the imaging probe 140 may weigh between 3 kg and 0.5 kg, or 0.3 kg, or 0.2 kg in some embodiments. For example, the imaging probe 140 may weigh between 6 lbs. and 3 lbs., or 2 lbs., or 1 lbs., in some embodiments.

The OCT imaging system 100 including the imaging probe 140, the console 130 and the fiber 103 may be carried by the users in a small carrying case with a handle, for example, that is less than 600 mm×400 mm×300 mm and weigh less than 15 kg or in another convenient manner due to its compactness. In some embodiments, for example, the carrying case is between (600 mm and 300 mm)×(400 mm and 200 mm)×(300 and 150 mm). Also, the carrying case weighs between 15 kg and 10 kg or 5 kg, in some embodiments. Sizes outside these ranges for the OCT imaging system 100 and the carrying case are also possible. Various embodiments may be easily operated by the operators with little training.

The imaging probe 140 of the OCT imaging system 100 may have a main portion 101, for example, a handle portion constructed to be in a cylindrical shape to allow easy grabbing by one hand and usable as a handle. The main portion is extending longitudinally from the distal end 129 of the probe housing. The main portion may be as disclosed in detail in U.S. patent application Ser. No. 14/220,005, which is hereby incorporated herein by reference in its entirety. The users may precisely adjust the position/angle of the imaging probe 140 with one hand, freeing another hand to work on other tasks, for example, opening the eyelids of the patient with the fingers. The first imaging module (for example, the color imaging module) can be disposed inside the main portion 101.

The OCT imaging module 110 can be disposed on a side portion or a top portion 110a, which is attached to the main portion 101. In some embodiments, the imaging probe 140 can comprise a handgrip with a bump. The handgrip portion with a bump may be as disclosed in U.S. patent application Ser. No. 14/312,590, which is hereby incorporated herein by reference in its entirety. The part of OCT imaging module 110 can be positioned inside the top portion 110a. In some embodiments, the diameter of the cylindrical handgrip can be 20 mm to 80 mm, for example, the diameter of the cylindrical handgrip can be 30 mm to 50 mm. In some embodiments, the length of the cylindrical handgrip can be 60 mm to 300 mm, for example, the length of the cylindrical handgrip can be from 100 mm to 200 mm. The housing of the OCT imaging module 110, for example, the top portion 110a, can have a width of from 10 mm to 50 mm, a height of from 10-80 mm and a length of from 20 mm to 100 mm. For example, the top portion 110a, which can be the housing of the OCT imaging module 110, can be about 20-30 mm wide, 30-50 mm high and 40-60 mm long. Values outside the above ranges are also possible.

Captured images may be transferred to other computing devices or internet based devices, like storage units, through wired or wireless communication systems. The imaging probe 140 can further comprise a processor to control the first imaging module and the OCT imaging module. The imaging probe 140 can further comprise a wireless transmitter and a wireless receiver to communicate with the console 130 or other computing devices or internet based devices wirelessly. The imaging probe 140 can comprise a display 106 with a user input interface such as a touch screen monitor mounted at the top of the main portion 101. The display 106 can be configured to display a first image from the first imaging system and a second cross sectional image from the OCT imaging module 110 simultaneously. In some embodiments, the imaging probe 140 is powered by a battery. Also in various embodiments, live images may be displayed on the touch screen monitor or a larger display monitor that receives data from this imaging probe 140 in real time.

The OCT imaging system 100 may be used as a disease screening or medical diagnosis device for the ophthalmic applications. It may be used in remote rural areas where traveling to the eye care facilities is not convenient. The OCT imaging system 100 may also be used as a portable medical imaging device to view other parts of the body (for example, ear or skin, etc.) for other medical needs such as ENT, dermatology, etc. Furthermore, the OCT imaging system 100 may have applications in areas other than medical applications, for example, for security screening applications where the images from the posterior/anterior segment of the eye may be used for the personal identification purpose. The OCT imaging system 100 may also be used to view a variety of objects including animals. The OCT imaging system 100 may also have other industry applications.

In some other embodiments, an optical coherence tomography (OCT) imaging system can only include an imaging probe. The handheld imaging probe can comprise an optical window, a first imaging module (for example, a color imaging module), a second imaging module (an OCT imaging module) and a console. The console can be disclosed inside the imaging probe. The console can be a miniature console integrated with the imaging probe. The console can comprise an OCT engine and a scanning mirror controller. The OCT engine can comprise an interferometer, a second light source (for example, an OCT light source with wavelength from 800 nm to 1100 nm), a light detector, a data acquisition system, and a processor. The console can be connected to the second imaging module by one or more fibers disposed inside the imaging probe.

FIG. 1B is a perspective view of an optical coherence tomography (OCT) imaging system 100b having an imaging probe 140b according to some other embodiments of the disclosure. As shown in FIG. 1B, the OCT imaging system 100b can comprise an endoscope 140b, which is a handheld imaging probe. The endoscope 140b, the handheld imaging probe, can comprise a housing with a distal end 129b and an OCT imaging module 110b. The OCT imaging module 110b is dispose within the endoscope 140b.

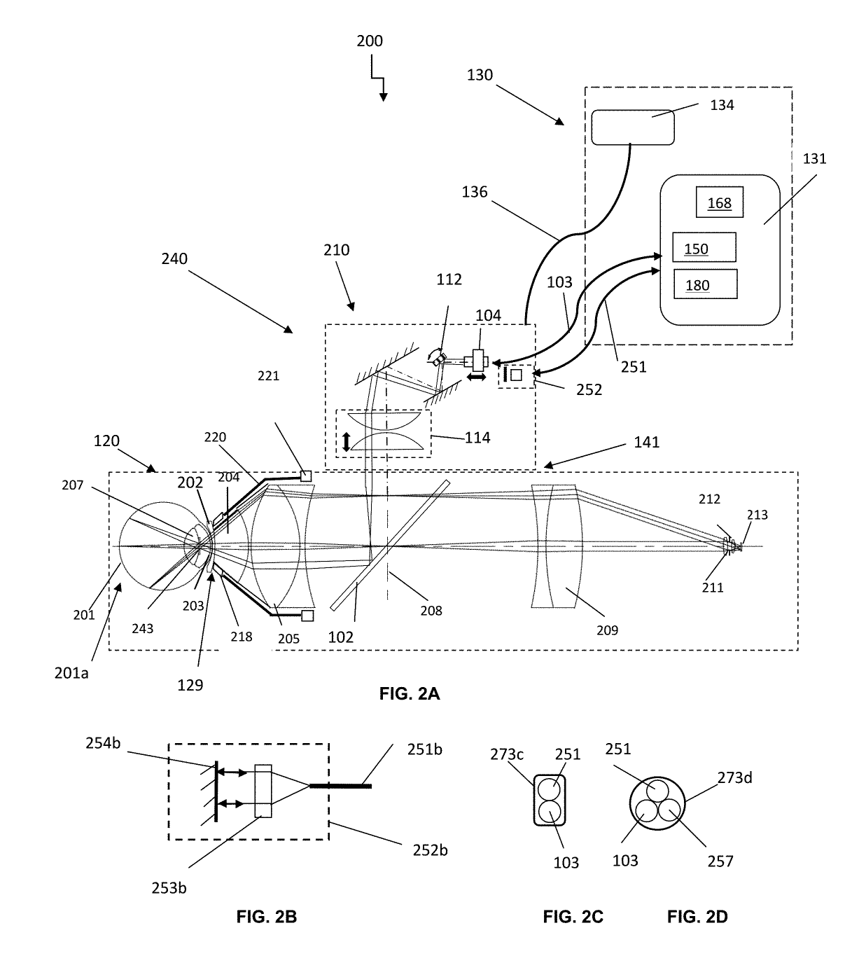

FIG. 2A schematically illustrates an example optical design of an ultra-wide FOV OCT imaging system 200 where a posterior segment 201 of an eye 201a is imaged according to one embodiment of the disclosure. Referring to FIG. 2A, the OCT imaging system 200 can have a remote imaging probe 240. The remote imaging probe 240 can have a probe housing 141 with a distal end 129. The term “remote” is used to mean the imaging probe is connected to the console by a cable, not integrated. The length of the cable can be 5 mm, 50 mm, 100 mm, 1000 mm, 2000 mm, 5000 mm, 8000 mm or any values therebetween. The terms “distal” and “front” are used interchangeably in this disclosure. Both “distal end” and “front end” means an end closest to the cornea 203 of the eye 201a.

The imaging probe 240 can include an optical widow 202 configured to be in contact with a cornea 203 of an eye 201a. The imaging probe 240 can include a first imaging module 120 and a second imaging module 210. The first imaging module 120 is configured to form a first image of the eye 201a. For example, the first imaging module 120 can be a two dimensional (2D) color imaging module. The first imaging module 120 can include a first light source 221 and a light conditioning element 218, where the first light source 221 is configured to provide a first light beam and the light conditioning element 218 is configured to direct the first light beam through the optical window 202 to the eye 201a. The second imaging module 210 is configured to form a second image of the eye 201a. For example, the second imaging module 210 can be an OCT imaging module to provide a volumetric image of the eye 201a, such as a cross-sectional view, an en face view, or a three-dimensional view of the eye. The second imaging module 210 can include a scanning mirror 112 configured to receive a sample arm portion of a second light beam from a second light source 150, to scan the sample arm portion, to receive reflected light of the sample arm portion and to reflect back the sample arm portion. The second light source 150 can be disposed on a console 130, or in the imaging probe. The second light beam of the second light source 150 can include the sample arm portion and a reference arm portion. The imaging probe 240 can further include a reflection module 252. The reflection module 252 is configured to receive the reference arm portion of the second light beam from the second light source 150 and to reflect back the reference arm portion to the interferometer module 180. The ultra-wide FOV OCT imaging system 200 can further include a beam splitter 102 configured to transmit the first light beam and to reflect the sample arm portion of the second light beam.

As shown in FIG. 2A, the ultra-wide FOV OCT imaging system 200 can further include the console 130 and a cable coupled between the console 130 and the imaging probe 140. The console 130 can include a scanning mirror driver 134 and an OCT engine 131. The OCT engine 131 can comprise an OCT interferometer 180, the second light source 150 or the OCT light source 150, and a processor 168. For example, the OCT interferometer 180 can be a fiber optical interferometer. The OCT light source 150 can comprise a swept light source in some embodiments. The OCT interferometer 180 can be the core of the OCT engine 131.

The interferometer 180 is configured to receive the reflected light from the second imaging module 210 and to generate data of the interference between the sample arm portion and the reference arm portion of the second beam. The processor 168 is configured to process the data from the interferometer 180 and to generate the second image, which is the OCT image. The cable can include a first fiber 103 and a second fiber 251, where the first fiber 103 is configured to transmit the sample arm of the second light beam to the second imaging module 210 in the imaging probe 240 and to transmit reflected light of the sample arm from the second imaging module 210 to the interferometer in the console 130; where the second fiber 251 is configured to transmit a reference arm of the second light beam from the second light source to the second imaging module 210 in the imaging module 240 and to transmit reflected light of the reference arm from the second imaging module 210 to the interferometer in the console 130.

The imaging probe can 240 comprise a probe housing 141 with a distal end 129, where the optical window 202 is disposed at the distal end 129. The first imaging module 120 can comprise a first light source 221 disposed inside the housing 141, where the first light source 221 can have a first wavelength range. The optical window 202 can have a concave front surface with a radius of curvature of from about 3 mm to about 18 mm. The optical window 202 can be in contact of the cornea 203 directly, or indirectly through a disposable cap. The index matching gel can be applied between the optical window 202 and the cornea 203. The optical window 202 is configured to match a radius of curvature of the cornea 203 of the eye. In order for the first imaging module 120 to have a wide field of view, the use of the optical index matching gel between the optical window 202 and the cornea 203 helps to eliminate significant amount of optical aberrations originated from the cornea 203 of the eye. The detail of the disposable cap may be as disclosed in U.S. patent application Ser. No. 15/007,101, titled: “A DISPOSABLE CAP FOR AN EYE IMAGING APPARATUS AND RELATED METHODS” and filed on Jan. 26, 2016, which is incorporated by reference in its entirety. The term “in contact” is defined herein to include in contact directly, in contact through an index matching gel, and in contact through a disposable cap.

In use, the optical window 202 may be placed in contact with the cornea 203 with slight pressure to obtain a wide field of the view of the retina 201 through the pupil. Accordingly, the optical window 202 may have a front concave surface with a radius of curvature closely matching a curvature of the cornea 203 of the eye. In some embodiments, the front concave surface of the optical window 202 has a radius of curvature of between 3 mm and 18 mm. For example, the front concave surface of the optical window 202 can have a radius of curvature of between 5 mm and 15 mm. Values outside the above range are also possible. The radius of curvature of the concave front surface of the optical window 202 can be changed depends on the need of the application.

In general, the first imaging module 120 can be configured to capture a color image of an eye, or a photograph of the eye. The first imaging module 120 can have a first illumination path and a first imaging path, where the first imaging path is separated from the first illumination path. The first imaging module 120 can comprise a first light source 221 disposed inside the imaging probe 140 to illuminate the eye through the independent illumination optical path. The first imaging module 120 can comprise imaging and focusing optics and an image sensor 213. The image sensor 213 is configured to receive reflected light from the eye through the separated imaging path in the first imaging module 120. The first illumination path is outside the first imaging path, and the first imaging path does not overlap with the first illumination path. The separation of the illumination path and the imaging path can reduce the scattered light from the cornea and widen the field of view of the first imaging module 120 when imaging the posterior segment of the eye 201a, the retina 201. Otherwise, the scattering light from the cornea 203 is much stronger than the reflected light from the retina 201, and the achievable field of view of the first imaging module 120 may be limited.

The first imaging module 120 can further comprise a light conditioning element 218. The light conditioning element 218 can comprise a multi-segment (e.g., reflective and/or refractive) surface and is positioned behind the peripheral portion of the optical window 202. The light conditioning element 218 is configured to receive a first light beam from the first light source 221 and directional control the first light beam to illuminate the eye through a first illumination path in a desired way to result in a wide field of view of the first imaging module 120. Further details of the light conditioning element 218 are disclosed in U.S. Pat. No. 9,155,466, which is incorporated by reference herein in its entirety.

To obtain high quality images, proper illumination is provided through the proper portion of the natural opening of the eye 201a while avoiding the first imaging path. In particular, illumination is provided through the peripheral regions of the eye pupil while the light scattered back from the posterior segment 201, the retina, of the eye 201a will pass through the central portion of the eye pupil which eventually forms the first image by the image sensor 213. This approach reduces backscatter from the central portions of the pupil, which would degrade the image of the retina obtained by light reflected from the retina also passing through the pupil. Since the eye is a complicated biological organ with its own special optical systems, the scattering and reflection from the eye in combination with its small aperture cause significant difficulties in obtaining a high quality image. In particular, the reflection and scattering from the eye cause glare and haze, which obscures the images acquired by an eye imaging apparatus. Thus the images from the posterior segment of the eye with a wide field of view often exhibit a layer of strong haze or glare. This problem is especially acute for the patients with dark pigmentation in the eyes. Providing illumination through certain regions of the eye as described herein, however, can reduce this backscatter and reflection and the resultant haze and glare. Therefore, the first imaging module 120 can further achieve a wide field of view. In some embodiments, the first imaging module 120 can have a field of view of at least 120 degrees, no more than 180 degrees. In some embodiments, the first imaging module 120 can have a field of view of at least 130 degrees, no more than 180 degrees. In some embodiments, the first imaging module 120 can have a field of view of 130 degrees.

The first imaging module 120 of the OCT imaging system 200 can comprise an imaging lens 204. The imaging lens 204, which may include one or multiple lens elements, is positioned behind the optical window 202 and optically aligned with the optical window 202. The optical axis of the optical window 202 and imaging lens 204 may, for example, be substantially aligned with the optical axis of the eye in some cases but not all. For example, the practitioner may examine the eye in a manner that the optical axis of the first imaging module 120 is substantially aligned with the optical axis of the eye; however, in some cases, the practitioner may tilt the handheld imaging probe 240 such that these axes are not aligned. Although the radius of the curvature for the frontal surface of the optical window 202 is chosen to closely match that of the cornea, the back surface of the optical window 202 may be flattened out slightly depending on the design of the first illumination path. The optical window 202 may be made from the same or different optical materials as the imaging lens 204. The curvature of the frontal surface of the imaging lens 204 may be the same as that of the back surface of the optical window 202, or different. The back surface of the imaging lens 204 may be either spherical or non-spherical to obtain desired result for the images. In some embodiments, a small gap of air or other material is placed between the optical window 202 and the imaging lens 204, although the two optical components may be in contact in certain areas or even bonded or affixed together with adhesive.

The first imaging module 120 can comprise a first focusing lens 211 to adjust a first focus of the first imaging module 120, and the image sensor 213 configured to receive a first image of the eye through the first imaging path. The first imaging path is separated from the first illumination path. The first imaging path does not overlap with the first illumination path. The light conditioning element 218 is configured to direct substantially all the light exiting the light conditioning element 218 outside an entrance pupil 243 of the first imaging system. Further details of the first imaging module 120 are disclosed in U.S. Pat. No. 9,155,466, which is herein incorporated by reference in its entirety.

In some embodiments, the first imaging module 120 may further include a first set of relay lenses 205 configured to form a secondary image of the eye near a back focal plane of the first set of relay lenses on a secondary image plane 208, a second set of relay lenses 209 configured to project the secondary image to infinity with a front focal plane positioned near the back focal plane of the first set of relay lenses. In various embodiments, a first focusing lens 211 is positioned near the back focal plane of the second set of relay lenses and configured to deliver light from the eye to the image sensor 213. For example, the first focusing lens can be a set of miniature focusing lenses. In some embodiments, the image sensor 213 and the first focusing lens 211 can are disposed in a miniature camera module. The miniature camera module comprising the first focusing lens 211 and the image sensor 213 has a format no more than 1/2.2 inches or 1/3.2 inches with a focal length of about 4 mm or less, for example between about 4 mm and 2 mm or 4 mm and 3 mm, etc. The view angle for the miniature lens or lenses may be 75° or less with a sensor appropriately sized based, for example, on the focal length of the miniature lens. The camera module, which includes the sensor 213 and the first focusing lens or lenses 211, is about 8.5×8.5 mm, or between 10 mm×10 mm and 5 mm×5 mm or smaller, for example. In some embodiment, for example, the first focusing lens or lenses 211 have aperture sizes between about 0.8 mm and 1.5 mm while the first and second relay lenses 205, 209 have aperture sizes of about 20 mm, for example between about 30 mm and 10 mm or 25 mm and 15 mm in some embodiments. The first imaging module 120 may gather light reflected from the posterior segment or more specifically the retina 201 of the eye 201a. The light passes through the center of an iris opening and the crystalline lens 207 of the eye, and forms a real image (of the posterior segment or retina) at the secondary image plane 208. As discussed above, the imaging lens 204 may include single or multiple lenses, with spherical or non-spherical surfaces. In some embodiments, the secondary image plane 208 is located near the back focal plane of lens 205. In some embodiments, a relay lens 209 may be used to project the image from the secondary image plane 208 to infinity when the front focal plane of the lens 209 is also placed near the secondary image plane 208. The image sensor 213, either in form of CCD, CMOS or other types, with its own miniature focusing lenses 211, may be positioned near the back focal plane of the lens 209 along the optical axis of the first imaging module 120. The miniature lenses 211 may include multiple optical lenses. In some embodiments, the image sensor 213 has an active area that is about 6.2 mm×4.6 mm or, for example, between about 8 mm and 4 mm×6 mm and 3 mm or between about 7 mm and 5 mm×5 mm and 4 mm. Accordingly, in various embodiments the active areas of the sensor 213 are about ¼ of the aperture size of the relay lenses 205, or for example between about 0.4 and 0.2 or 0.5 and 0.1 the size thereof. In various embodiments, the first focusing lens or lenses 211 are built with a circular optical aperture (iris) 212, which may be located between the first focusing lens or lenses 211 or formed by an aperture plate in front of the first focusing lens or lenses 211. In certain embodiments such location of the optical aperture 212 reduces optical aberration. The first focusing lens or lenses 211 may not only relay the image of the retina 201 to the image sensor 213, but also form an entrance pupil 243 for the first imaging module 120 near the anterior surface of crystalline lens 207 when the aperture 212 becomes the aperture of the first imaging module 120. This special arrangement helps to eliminate significant amount of scattering light from the anterior chamber of the eye and the optical elements in the first imaging module 120.

In some embodiments, the optical lenses in the first imaging path of the first imaging module 120 can be achromatized for the light beams within the wavelength range of the first light source 221. The optical lenses forming the first imaging module 120, from the distal end of the first imaging module, which includes the contact optical window 202, to the front of the imaging sensor 213, which includes the focusing lens 211, can be achromatized for the light beams of multiple wavelengths within the wavelength range of the first light source 221 in the first imaging module 120. Additional considerations may be needed in order to compensate the residual chromatic aberrations from the eye itself. Here the achromatization means the design to minimize the optical aberrations for multiple light wavelengths within the working wavelength range, not just one wavelength, as well as for the full field of view of the first imaging module 120. For example, the optical lenses forming the first imaging module 120 can be configured to minimize the optical aberrations at wavelengths of 470 nm, 550 nm and 650 nm for the working light wavelength range of 450 nm to 700 nm, and minimize the aberrations for the field of view from 0 degree (on optical axis) to 130 degree as well.

The first imaging module 120 can comprise a first illumination sub-module, which comprises the first light source 221, optical fiber bundles 220, the light conditioning element 218, and a periphery portion of the optical window 201. The first illumination sub-module forms the first illumination path, which is separated in space from the first imaging path. The first light beam may be emitted from the light source 221 and injected into the optical light conditioning element 218 positioned behind the peripheral portion of the optical window 202 though optical fiber bundles 220. The first illumination path is extending longitudinally forwardly and radially inwardly from the first light source 221, to optical fiber bundles 220, to the light conditioning element 218, to the periphery portion of the optical window 202, and to the posterior segment 201, the retina 201, of the eye 201a for posterior imaging. The first imaging module 120 can comprise a first imaging sub-module, which comprises a central portion of the optical window 202, the imaging lens 204, the first relay lens 205, the second relay lens 209, the first focusing lens 211 and the image sensor 213. The first imaging module forms the first imaging path for the reflected light from the eye. The first imaging path is extending longitudinally backwardly from the retina 201 to the central portion of the optical window 202, to the imaging lens 204, to the first relay lens 205, to the second relay lens 209, to the first focusing lens 211, and to the image sensor 213.

The first light source 221 can be a visible light source, for example, with a wavelength from 450 nm to 700 nm in some embodiments. The first imaging module 120 can be used to obtain a color full field image of the eye. The color full field image is preferred by doctors since it provides more information than a black and white image. However, in order to obtain a color image by using a visible light source, the dilation of the eye is required. In some other embodiments, the first light source 221 can be a near infrared (NIR) light with a wavelength range from 700 nm to 840 nm. When the first light source 221 is a NIR light source, the eye does not need to be dilated, which may make a child more comfortable during the OCT imaging session. The NIR light beam of the NIR light source has less scattering than the visible light beam because the longer wavelength light cause less scattering. Therefore, the OCT imaging system may get better and clearer OCT images by using the NIR light source than by using the visible light source.

Referring to FIG. 2A, the imaging probe 140 can comprise the second imaging module 210, which is an OCT imaging module. The OCT imaging system 200 can comprise the OCT imaging module 210 (an integrated OCT imaging module, or a removable OCT imaging module), which can provide OCT imaging in concurrent with the color imaging at the same time. In some embodiments, the OCT imaging module 210 may is permanently integrated with the first imaging module 120. The second imaging module 210 can have a second illumination path and a second imaging path. The second imaging module 210 can comprise the scanning mirror 112, for example, a scanning MEMS mirror, configured to scan the sample arm portion of the second light beam from the second light source 150. A second light beam from a second light source 150, which is disposed in the console 130, can be carried to the OCT imaging module 210, through the optical fiber 103 to form a sample arm for the OCT interferometer 180. The second light source 150 can be an OCT light source. For example, the second light source can be a broadband light source with a wavelength range from 800 nm to 1200 nm that is disposed in the console 130. In some embodiments, the second light source 150 is a swept source laser, which can provide a fast scanning speed. The optical fiber 103 can be a single mode fiber, either in the form of regular fiber or polarization maintaining fiber. The polarization maintaining fiber can reduce the effect of the shift of polarization state for the emitting light when the fiber 103 is being bended or twisted during the operation. The details of the polarization maintaining fiber will be discussed below. An optical coupling lens 104, which is acting as a collimator, is mounted in an either mechanical or electrical focus adjustment mechanism. The optical coupling lens 104 can be used to form a collimated light beam.

The scanning MEMS mirror 112 is disposed outside the first illumination path and the first imaging path. The OCT imaging system 200 can comprise a beam splitting dichroic mirror 102, which is a beam splitter, disposed in the first imaging path and configured to transmit the first light beam and reflect the sample arm portion of the second light beam, and a second focusing lens to adjust a second focus of the second imaging module 210. The beam splitting dichroic mirror 102, which can be configured to reflect light in the wavelength longer than 700 nm, can be inserted into the first imaging path of the first imaging module 120. The first imaging module 120 provides color imaging capability in the visible light spectrum, from 450 nm to 700 nm. The first light beam of the first imaging module 120 can pass through the beam splitter 102 while the second light beam from the second light source 150 for the OCT imaging module 210 can be reflected almost entirely. Some details of the second imaging module are disclosed in U.S. patent application Ser. No. 15/186,402, which is herein incorporated by reference in its entirety.

The OCT imaging module 210 can comprise a second illumination path and a second imaging path. The OCT imaging module 210 can use some of the same optical components used in the first imaging module 120 to perform its illumination and imaging functions. The OCT imaging module 210 can be configured to construct a cross-sectional view of the eye. The OCT imaging module 210 can comprise the beam splitting dichroic mirror 102 which can be configured to split the light beam in the imaging path for first imaging module 120 and OCT imaging module 210, and a scanning mirror, for example, a scanning MEMS mirror 112. The mirror driver or drivers can be connected to a scanning MEMS mirror controller 134 disposed in the console 130 by the data link 136. The OCT imaging module 210 and first imaging module 120 share the same optics from the beam splitting dichroic mirror 102 to the distal of the optics toward the patient's eye. For example, the OCT imaging module 210 and first imaging module 120 share the beam splitting dichroic mirror 102, the first relay lens 205, the imaging lens 204, and the optical window 202 as shown in FIG. 2A. The beam splitting dichroic mirror 102 can be disposed in the first imaging path and configured to transmit the first light beam of the first light source 221 and reflect the first portion of a second light beam from the second light source (not shown). In some embodiments, the scanning MEMS mirror 112 can be configured to scan a first portion of the second light beam from the second light source, where the scanning MEMS mirror 112 disposed outside the first illumination path and the first imaging path. The first imaging module 120 can be configured to provide registry of an imaging location of the OCT imaging module 210.

The OCT imaging module 210 can comprise a second focusing lens or lenses 114 configured to perform focus adjustment for the OCT imaging module 210. The OCT imaging module 210 can further comprise one or mirrors to manage the optical path of the second beam as shown in FIG. 2A. The second light beam can be focused by the second focusing lens or lenses 114, through the beam slitting dichroic mirror 102, on to an area near the secondary imaging plane 208 of the first imaging module 120. From there, the second light beam can be directed by the imaging optics (for example, the optical window 202, the imaging lens 204 and the first relay lens 205) in the first imaging module 120 to the targeted portion of the eye 201a, for example, the posterior segment 201. The imaging optics along this shared optical path can be designed and achromatized for working in both the wavelengths for visible first imaging module 120 and OCT imaging module 210. Therefore, the first imaging 120 and OCT imaging module 210 can share the same or approximately same secondary image plane 208 as shown in FIG. 2A. The optics for the OCT imaging path can be also designed such that the scanning mirror 112 can be located in a plane as an optical conjugate for the entrance pupil 243 of the first imaging module 120. The entrance pupil 243 can be located near the iris plane of the eye 201a, which is separated from the illumination light path of the first imaging module 120 and located near the central portion of the eye iris. In some embodiments, a real image of an aperture of the OCT imaging module 210 is positioned near an anterior surface of the crystalline lens of the eye 201a when the optical window 202 is in contact of the cornea 203. When the scanning mirror 112 is rotated, the scanning mirror 112 can direct the focused light beam to different locations in the posterior segment 201 of the eye 201a with different type of scanning patterns, the light beam can illuminate different parts of posterior segment 201 of eye 201a in a timely and consecutive fashion.

Because the first imaging module 120 has its own focusing mechanism which can activated when the posterior segment or retina 201 is out of focus, both the first image module 120 and the OCT image imaging module 210 can be configured to be focused at same time. To do that, part of the focusing lens group 114 can be configured to be movable, which helps to re-focus the OCT light beam on to the target if necessary. The movable focusing lens group 114 can be driven electrically with the driver electronics built on the board of driver electronics for scanning mirror 112 or manually. When the focusing lens group 114 is driven electrically, the motion of the focusing lens group 114 can be configured to synchronize with the motion of the focusing lens 211 of the first imaging module 120 such that the focusing action for both imaging modalities could be performed at same time with just one action. The focus of the OCT light beam can also be achieved by moving the optical couple lens 104 with its driving mechanism electrically with the driver electronics built on the board of driver electronics for scanning mirror 112, or manually.

In some embodiments, the OCT imaging system 200 can further comprise a third light source (not shown), which is an aiming light. The third light source is disposed in the console 130. A beam combiner (not shown) can be also disposed in the console 130, and the beam combiner is configured to couple both the second light source 150 and the third light source to the handheld imaging probe 240 through the optical fiber 103. The light from the optical fiber 103 not only includes the second light beam from the broadband light source 150 for OCT imaging, but also a third light beam as the aiming light with narrowband wavelength range. In some embodiments, the wavelength of the third light source can be between the second light source 150 and the first light source 221 for color imaging. For example, the aiming light source can have a wavelength of 680 nm, 700 nm or 740 nm. The third light beam can have a third illumination path and a third imaging path, wherein the third illumination path is the same as the second illumination path of the OCT imaging module and the third imaging path is the same as the first imaging path of the first imaging module. The beam splitting dichroic mirror 102 can be configured to partially reflect and partially transmit the third light beam. The aiming light beam is relayed to the posterior segment 201 of the eye 201a with the same optics of the OCT imaging module 210 and focused as at exactly the same spot, while the reflected aiming light beam is partially transmitted through the beam splitter 102 and then eventually projected to the image sensor 213 by the same optics as in the first imaging path.

The track of the aiming light beam is then shown on the first image, which not only provides visualization of the OCT scanning pattern in real time, but also a focusing status of the OCT scanning beam on the posterior segment 201. During the process of adjusting the first focus of the first imaging module 120 to give a clear color image, the linewidth of the aiming light beam seen on the image is also changed. The linewidth of the aiming light beam then can be measured in real time from the color image and provides feedback for the adjustment of the second focus for OCT imaging module. The feedback of the linewidth of the aiming light beam can be used to control the second focusing lens, for example, and the movable lens or lenses 114, or the coupling lens 104. As the result, the focus for both the first imaging module 120 and the OCT imaging module 210 can be synchronized in real time. Thus, the first adjustment of the first focus lens 211 and a second adjustment of the second focus lens are synchronized through the feedback of the linewidth of the aiming light beam.

As discussed above, the optical lenses in the first imaging path of the first imaging module 120 can be achromatized for the light beams within the wavelength range of the first light source 221. Because all optical materials have their unique properties of color dispersion, if the first imaging module 120 is not achromatized, then the images formed by different colors of light beams will not be superimposed into one image on the imaging sensor 213, causing reduction in contrast of the image significantly. The prior art has not adequately recognized that OCT imaging systems have even more severe problems of color dispersion than the conventional imaging systems. In general, an OCT imaging system comes with a broadband light source which can have an optical wavelength range spanning up to 100 nm. If the OCT imaging system is not achromatized, the light beams from different wavelengths, even if they traveled through the same optical pathway, will exhibit different total optical paths in the sample arm and result in different interference patterns when interfered with the reference arm in the fiber interferometer. Most of the conventional OCT imaging systems is trying to solve the problem by using a compensation module in the reference arm. For example, the compensation module can contain the same materials of the same thickness in the sample arm. However, for the OCT imaging systems, the light beams at different field of view (view angles) can also have different total optical paths even though the light beams go through the same optical materials. In general, the lenses have different thickness from their centers to the edges. When the light beams pass through different portions of the lenses, the total optical paths have different percentage of space (air) and glass mixtures for different field of view in the full imaging system. Although the dispersion compensation module is a fixed one, which can be designed to match the glass type and thickness for the light beam along the optical axis in the imaging path, the compensation module may not be correct for the light beam traveling through different part of the lenses, such as off-axis light beams. Moreover, the eye has optical dispersion which is not corrected. The lens in the imaging path has to be achromatized such that not only the aberrations caused by the lens materials are compensated, but also the optical dispersion from the eye is compensated.

In some embodiments, the optical lenses in the second imaging path of the OCT imaging module 210 can be achromatized for the light beams within the wavelength range of the OCT light source 250 for optical dispersion, from the eye all the way to the front end of the fiber 103. The optical lenses forming the OCT imaging module 210 from the distal end of the second imaging path, which includes the optical window 202, to the front end of the optical fiber 103, which is the optical coupling lens 104, can be achromatized for the light with wavelengths used in the OCT imaging module. The optical lenses in the second imaging path are further configured with additional consideration for the need to compensate the residual chromatic aberrations from the eye itself. Here the achromatization means the design to minimize the optical aberrations for multiple light wavelengths within the working wavelength range, not just one wavelength, as well as for the full field of view of the OCT imaging module 210. For example, the optical lenses in the second imaging path are configured to minimize the optical aberrations at wavelengths of 1000 nm and 1080 nm for the working light wavelength range of 980 nm to 1100 nm, as well as for the field of view from 0 degree (on optical axis) to 130 degree.

In some other embodiments, the achromatization can be performed to minimize the axial color aberration for the selected light wavelengths for the full field of view of the OCT imaging module 210. The optical path difference for the light beams transmitting within the same portion of the optical lenses but with different optical wavelengths can be minimized to an allowable amount, and the same allowable amount of optical path difference can be applied to light beams transmitting through different portions of the optical lenses. For example, the light path difference for the light beams with optical wavelengths of 1000 nm and 1090 nm can be minimized to 2 nm for light beams transmitting along the optical axis and minimized to around 2 nm for light beam with the field of view of 130 degrees.

In some embodiments, the optical lenses in the first imaging path of the first imaging module 120 can be achromatized for optical dispersion for the wavelength range of the first light source 221, from the eye all the way to the imaging sensor 213. Therefore, the optical lenses in the shared portion of the first imaging path and the second imaging path, from the distal end 129 of the imaging probe 240 to the dichroic beam splitter 102, can be achromatized for optical dispersion of the working optical wavelengths of both the first imaging module 120 and the OCT imaging module 210. The optical lenses shared by both the first imaging module 120 and the OCT imaging module 210, for example, the optical contact window 202, the imaging lens 204 and relay lens 205, can be achromatized for both optical wavelength bands in the first imaging module 120 and the OCT imaging module 210.

As shown in FIG. 2A, the imaging probe 240 is connected with the console 130 through the optical fibers 103 and 251 which often are identical or similar in their construction and optical properties. The console 130 can comprise the interferometer module 180 inside. The fiber 103, which can be a sample arm beam fiber, can guide the light beams from the interferometer module 180 to the imaging probe 240 and illuminate the eye, for example, retina (target) 201, which is to be imaged. The fiber 103 can further receive light reflected from the retina 201 and eventually guide the reflected light beams back to the interferometer 180. The fiber 251, which can be a reference beam fiber, can guide the light beams from the interferometer module 180 to the reflection module 252, and it returns the reflected light back to the interferometer module 180. For example, the reflection module 252 can be a simple reflective coating on the end surface of the optical fiber 251, where the end surface of fiber 251 is cut to be normal to the optical axis of the fiber 251 and polished to have optical quality before a reflective coating is added, or a light reflector (mirror) is added against or close to the end surface of the fiber 251.

FIG. 2B illustrates another embodiment of a reflection module 252b in an interferometer of the OCT imaging system. As shown in FIG. 2B, a collimating lens 253b is placed with its back focusing plane located at or near the end of the fiber 251b and with its optical axis aligned with the optical axis of the fiber 251b, and thus forming a collimated light beam to illuminate a reflective surface 254b. The reflector (mirror) 254b can be placed to be perpendicular to the optical axis of the collimating lens 253b. The light reflected by the reflective surface 254b is then focused back, along the same optical path, to the end of fiber 251b and return to the optical fiber 251b in opposite transmission direction.

FIG. 2C illustrates an optical fiber cable 273c according to one embodiment of the disclosure. FIG. 2D illustrates another optical fiber cable 273d according to another embodiment of the disclosure. The fiber optical cable can comprise a plurality of optical fibers. As shown in FIG. 2C, the cable 273c can comprise optical fibers 103 and 251 inside, thus making the two fibers experiencing the same (or very similar) deformation when the cable 273c is stretched or bended during the use of the OCT imaging probe. In some embodiments, the optical fibers 103 and 251 can be placed next to each other and side by side and enclosed by a protective sheath 273, which not only protect the fibers from environment, but also keeps the fibers in fixed locations relative to each other even when the cable is stretched, twisted or bended. As sown in FIG. 2D, in another embodiment, additional fiber 257 can be placed within a sheath to form a more stable triangle style of construction, which is easier to construct.

Advantageously, the contact type OCT imaging system 200 can result in a super wide field of view. When the imaging probe 240 comes to in contact with the cornea 203 of the eye 201a directly or through a disposable cap, the OCT imaging system 200 can be used to image the posterior segment 201, or the retina, of the eye 201a, in the form of a contact microscope. For example, the contact OCT imaging system 200 can have the field of view, measured from the center point of the eye, that is 3 to 5 times of a conventional non-contact OCT imaging system. The microscope type optical design of the contact OCT imaging system 200 can result in a wider field of view than the telescope style optical design of the conventional non-contact OCT imaging systems for imaging the posterior segment 201 of the eye 201a.

The OCT imaging module 210 can have an ultra-wide field of view. In some embodiments, the contact OCT imaging system 200 can have the field of view, measured from the center point of the eye, of between 60 degrees×60 degrees to 120 degrees×120 degrees in a single volume acquisition. The field of view may in certain embodiments be at least 120 degrees×120 degrees and up to 180 degree×180 degrees in a single volume acquisition. In some embodiments, the field of view is at least 130 degrees×130 degrees but no more than 180 degrees×180 degrees in a single volume acquisition. In some embodiments, the field of view is at least 20 mm-30 mm by 20 mm-30 mm in a single volume acquisition.

Moreover, the contact OCT imaging system 200 is advantageous for pediatric application since the babies are often not following the instruction of the operators. For pediatric application, the non-contact type OCT imaging system does not work well at babies because the non-contact OCT imaging system need a cooperative object who can follow the instructions of the operator. The contact type OCT imaging system can overcome the problems of the non-contact system and work well with babies.

One of the problems of non-contact OCT imaging systems is the need for sophisticated automatic (or smart) adjustment for a reference arm. In general, an OCT imaging system has an axial resolution around 6 μm. The relative movement in axial distance between the OCT imaging system 200 and the eye of the patient causes the vertical motion in OCT images. The distance between the OCT imaging system 200 and the eye of the patient has to be kept constant to the range of tens of microns in order to see the live OCT images stabilized to an acceptable level. To keep the OCT images even in the picture frame, which is related to the working range of the axial distance, the distance has to be maintained to be within less than 1-2 mm. For a non-contact OCT imaging probe, which may be about 3 lbs. to 6 lbs. and away from the eye, it is very difficult to align the non-contact OCT imaging probe precisely with an eye of an infant or a small child. Conventionally, it typically takes about 20 minutes or more for doctors/operators to get alignment right and be able to take an OCT image since often the OCT images may only show up for a couple of seconds and move out. The eye of a child may move, and the hand of the doctor/operator may move as well. It is difficult to automatically compensate and stabilize such motions in the OCT images by the reference arm adjustment.

The contact OCT imaging system 200 can be advantageously to provide better stabilization of the OCT images during the operation and increase the signal-to-noise ratio of the OCT images than the conventional non-contact OCT imaging systems. The contact OCT imaging system 200 is placed in contact with the cornea of the eye; such arrangement can minimize the possible change of distance between the eye (more precisely the retina 201 for the posterior segment imaging) and the OCT imaging system 200 to a fraction of 1 mm, thus reducing the vertical motion in OCT images. Therefore, during an OCT imaging procedure, the OCT image is easily kept within the picture frame. As soon as the OCT imaging system 200 is in contact with the cornea 203, the doctors/operators may immediately see the live OCT image without the need to struggle for alignment. The live OCT image may still move in the picture frame constantly because the distance control is still not to the level of tens of microns. However, combining with high speed imaging technique, the quality of the OCT image can be significantly improved, and the time to acquire the OCT image can be significantly reduced.

In addition, the OCT imaging system 200 can minimize the lateral alignment requirement between the patient's eye and the optical system of the OCT imaging system 200. Because the shape of the contact optical window 202 is designed to fit closely with the shape of the cornea 203, it is easier to align the contact optical window 202 with the cornea 203 when they are in contact. The contact optical window 202 can also reduce the free motion of the eye ball too, and helps to stabilize images in its lateral motion. The OCT imaging system 200 can further minimize the optical aberration caused by either the misalignment of the eye with the OCT imaging system 200, or the aberration from the eye, by using the index matching gel in the space between the eye and the contact optical window 202. Such reduction helps to increase the signal to noise ratio of the OCT images because more light could be couple to the optical fiber from the eye when aberration is reduced. The contact optical window 202 can widen the field of the view of the OCT imaging system 200 and enable the imaging of peripheral area of the posterior segment 201, the retina, where the conventional non-contact type OCT imaging systems cannot reach.

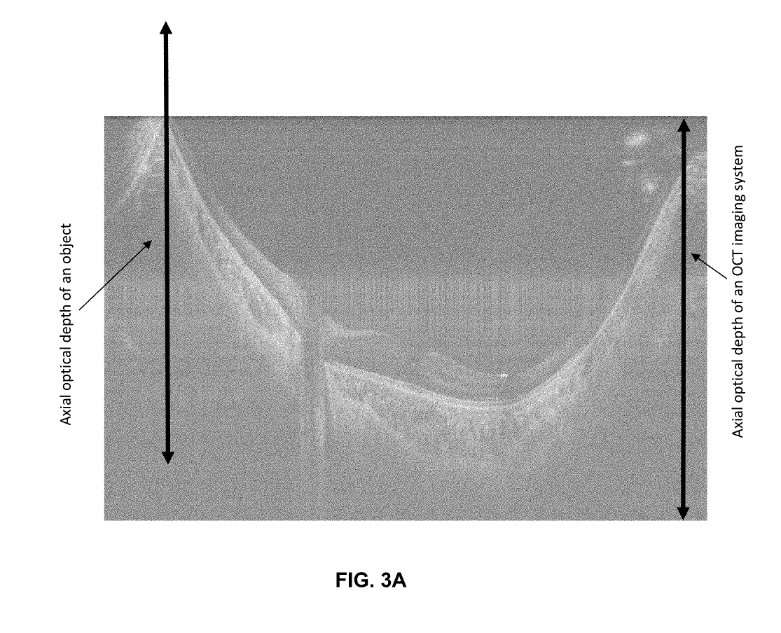

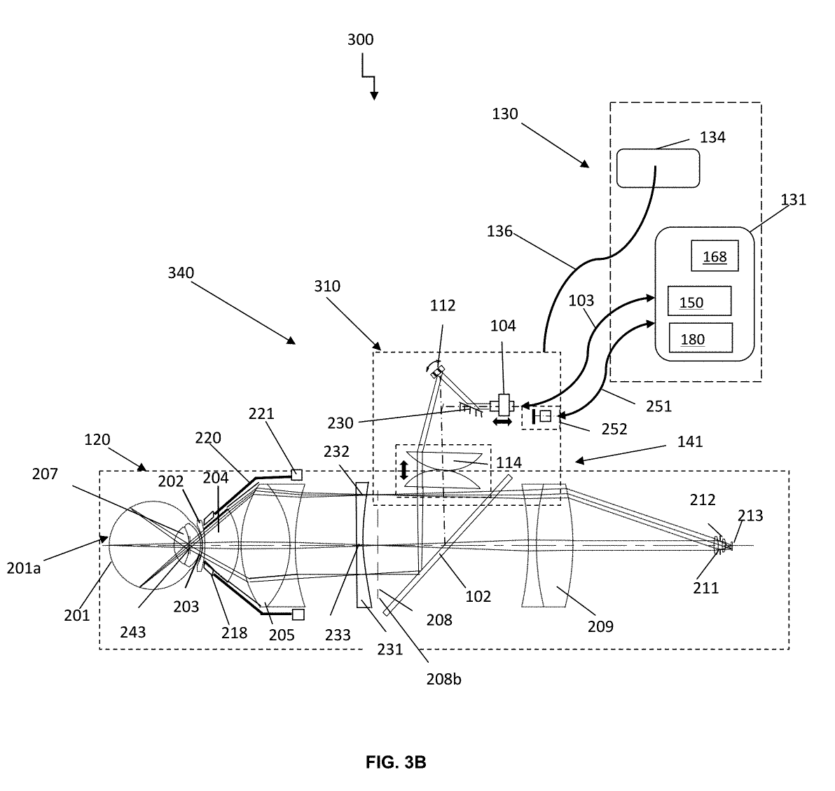

FIG. 3A illustrates an OCT image of a posterior segment of an eye, where a folding in phenomena is exhibited at a left-upper corner. In an ultra-wide FOV OCT imaging system, the object to be imaged, for example, a retina of the eye, can have an axial optical depth of the object exceeding an axial optical depth of the OCT imaging system. As known to the person skilled in the art, an optical path length (OPL) is defined as the product of the geometrical length of the path light follows through the system, and the index of refraction of the medium through which it propagates. Due to the curvature of the retina, different light beams to the retina will have different OPLs. Thus, there can be optical path length differences along an optical axis of the OCT imaging system between the different light beams on different parts of the object. For example, there can be a maximum optical path length difference along the optical axis between a first optical path length of the light beam on a center of the retina and a second optical path length on a peripheral region of the retina. The axial optical depth of the object is the maximum optical path length difference of light beams along the optical axis on different parts of the object. For example, the axial optical depth of the object can the maximum axial optical path length difference of light beams on different parts of the object for a central wavelength of the scanning wavelength range of the OCT light source. In some embodiments, the axial optical depth of the object is the maximum OPD of the object between a center and a peripheral region of the object along the optical axis. The axial optical depth of the OCT imaging system is an axial scanning range of an A-scan of the OCT imaging system. The axial optical depth of the OCT imaging system is determined by the coherence length of an OCT light source (a second light source of an OCT imaging system) and the design of the data acquisition system for A-scan of the OCT imaging system. Often the axial optical depth of the OCT imaging system is less than the coherent length of the OCT light source.

When the axial optical depth of the object is larger than the axial depth of the OCT imaging system, the lack of the axial optical depth of the OCT imaging system to cover the full axial optical depth of the object can either result in missing of important features in the OCT image of the object, or a portion of the features of the OCT image being folding into the area where such features do not exit, as shown in FIG. 3A. The lack of the axial optical depth of the OCT imaging system to cover the full axial optical depth of the object can result in a reduction of an actual achievable field of view of the OCT imaging system.

One of the solutions to the above problem is to reduce the axial optical depth of the object, which is the OPD of light beams from the center to the peripheral region of the object, to be less than the axial depth of the OCT imaging system, for example, at a central wavelength of the scanning wavelength range of the OCT light source. Typically, the axial depth of the OCT imaging system is about 3 mm. The OPD compensation is related to the correction of curvature of image field. The result of the OPD compensation is a flattened OCT image for the ultra-wide FOV of the OCT imaging system.

FIG. 3B is a schematic of an example optical design of an ultra-wide FOV OCT imaging system 300 with an optical path difference (OPD) compensator 231 according to some embodiments of the disclosure. The OPD compensator 231 can be disposed in an optical path of the sample arm portion of the second light beam from a second light source 150. The OPD compensator 231 can include a center 233 and a peripheral region 232, where a first optical path length of the sample arm portion along the center 233 is shorter than a second optical path length along the peripheral region 232. The OPD compensator 231 is configured to reduce an axial optical depth of the object, for example, the eye 201a, thereby extend the actual achievable field of view of the OCT imaging system 300, in order to solve the problem discussed above.

As shown in FIG. 3B, the OCT imaging system 300 can have a remote imaging probe 340. The imaging probe 340 can include an optical widow 202 configured to be in contact with a cornea 203 of the eye 201a. The imaging probe 340 can include a first imaging module 120 and a second imaging module 310. The first imaging module 120 is configured to form a first image of the eye. The first imaging module 120 can include a first light source 221 and a light conditioning element 218. The second imaging module 310 is configured to form a second image, an OCT image, of the eye 201a. The second imaging module 310 can include a scanning mirror 112 configured to receive a sample arm portion of a second light beam from a second light source 150. The second light source 150 can be disposed on a console 130, or in the imaging probe. The second light beam of the second light source 150 can include the sample arm portion and a reference arm portion. The imaging probe 240 can further include a reflection module 252. The reflection module 252 is configured to receive the reference arm portion of the second light beam from the second light source 150 and to reflect back the reference arm portion. The ultra-wide FOV OCT imaging system 300 can include a beam splitter 102 configured to transmit the first light beam and to reflect the sample arm portion of the second light beam.

The ultra-wide FOV OCT imaging system 300 can further include a console 130 and a cable. The cable can include a first fiber 103 and a second fiber 251, where the first fiber 103 is configured to transmit the sample arm of the second light beam to the second imaging module 310 in the imaging probe 340 and to transmit reflected light of the sample arm from the second imaging module 310 to the interferometer 180 in the console 130. The second fiber 251 is configured to transmit a reference arm of the second light beam from the second light source 150 to the second imaging module 310 in the imaging probe 340. In some embodiments, the second fiber 251 is further configured to transmit reflected light of the reference arm from the second imaging module 310 to the interferometer 180 in the console 130. In some embodiments, the cable further includes a third fiber (not shown). The third fiber is configured to transmit reflected light of the reference arm portion from the second imaging module to the interferometer. In some embodiments, the first fiber, the second fiber and the third fiber are closely fixed inside the cable such that external motion effects cause same changes in polarization direction and optical path lengths for the first fiber, the second fiber and the third fiber.

In order for the OCT imaging system 300 to achieve the ultra-wide FOV, the design of the imaging lens group, including lens 204, 205, 114, as well as the scanning mirror 112, can be optimized with the aim to reduce the full axial optical path difference (OPD) of the imaged object, for example, the retina 201 of the eye 201a, for light beams within the full field of view of the OCT imaging system 300 to be less than the axial depth range of the OCT imaging system 300. As discussed above, the axial depth range of the OCT imaging system 300 is determined by the coherence length of the OCT light source 150, which is the second light source 150. In some embodiments, the selection of optical materials for those lenses can be used for such optimization processes. In some embodiments, the optical architect of the OCT imaging system 300 can be configured to realize such optimization processes.

The OPD compensator 231 is introduced to further extend the achievable FOV of the OCT imaging system 300. As shown in FIG. 3B, a mirror 230 is configured to guide a collimated light beam, which is the sample arm portion of the second light beam of the second light source 150, from an optical coupling lens 104 to the scanning mirror 112. The collimated light beam is then reflected by the scanning mirror 112, and focused by a relay lens group 114, and reflected by the beam splitter 102, to a secondary image plane 208b (could be curved) of the second imaging module 310 (the OCT imaging module). In some embodiments, the secondary image plane 208b is configured to be both a secondary image plane 208 of the first imaging module 120 and the secondary image plane 208b of the second imaging module 310. In some embodiments, the secondary image plane 208 of the first imaging module 120 is closely located to the secondary image plane 208b of the second imaging module 310 within 1 mm, 3 mm or 5 mm, or any values there between. As the scanning mirror 112 rotates, the focused light beam, the sample arm portion of the second light beam of the second light source 150, would be steered across the entire secondary image plane 208b. Because the secondary image plane 208b is conjugated to the retina 201 of the eye 201a through the imaging lenses between the eye 201a and the beam splitter 102, the focused beam from the second light source 150 would be relayed to the retina 201 of the eye 201a as well.

Due to the curvature of the retina 201, different light beams exiting from an end of the first fiber 103 to the retina 201 will have different optical path lengths. When the light beam is steered from a center of the retina 201 to a peripheral region of the retina 201, in general, a first optical path length of the light beam along the center of the retina 201 would exhibits a longer axial optical path length than a second optical path length of the light beam along the peripheral region of the retina 201. As discussed above, for OCT imaging systems, the axial optical depth of the OCT imaging systems is limited by the coherent length of the light source. Particularly, for the OCT imaging system 300 with the ultra-wide field of view, the coherent length of the light source 150 may be shorter than such difference in the axial optical path lengths. For example, there can be a maximum OPD of the retina 201 along the optical axis between a first optical path length of the light beam on a center of the retina 201 and a second optical path length on a peripheral region of the retina 201. In some embodiments, the axial optical depth of the retina 201 is the maximum optical path length difference of the retina 201 between a center and a peripheral region along the optical axis. When the axial optical depth of the retina 201 is larger than the axial depth of the OCT imaging system 300, the lack of the axial optical depth of the OCT imaging system 300 to cover the full axial optical depth of the retina can either result in missing of important features in the OCT image of the retina 201, or a folding in phenomena as shown in FIG. 3A.

To compensate for the lack of the axial optical depth of the OCT imaging system 300 to cover the full axial optical depth of the retina 201, the OPD compensator 231 is disposed closely to the secondary image plane 208b (could be curved) of the second imaging module 310, in the optical path of the sample arm portion of the second light beam of the second light source 150. The OPD compensator 231 can be located closely the secondary imaging plane 208b of the second imaging system 310, for example, within 0.1 mm to 3 mm. In some embodiments, the secondary image plane 208 of the first imaging module 120 is within 5 mm to the secondary image plane 208b of the second imaging module 310. Because the OPD compensator 231 is located closely to both the secondary image plane 208b of the second imaging module 310 and the secondary image plane 208 of the first imaging module 120, the OPD compensator 231 has little impact on the image quality or optical aberrations on the images. The OPD compensator 231 can result in the flattening of the OCT image, which is advantageous to achieve the ultra-wide field of view.

The OPD compensator 231 can be made of transparent optical material, for example, glass, crystal or plastic. In some embodiments, the OPD compensator 231 has at least one concaved surface. The concave surface could be a spherical or aspherical surface. In some embodiments, the OPD compensator 231 can have a shape of double concave, or a planer-concave design. In some other embodiments, the OPD compensator 231 can have two flat planes with a lower index of refraction along a center and a higher index of refraction along a peripheral region. The first optical path length of the light beam passing through the center 233 of the OPD compensator 231 has a shorter axial optical path than the second optical path length of the light beam passing near the peripheral region 232 of the OPD compensator 231. As the result, the difference in the axial optical path lengths of the retina 201 can be reduced by the OPD compensator 231. The axial optical depth of the retina 201 corresponds to the difference in the axial optical path lengths between a first optical path length along the center and a second optical path length along the peripheral region. Therefore, the axial optical depth of the retina 201 is reduced and the actual achievable FOV of the OCT imaging system 300 is extended, or enlarged.

For example, the difference in optical path lengths, from the center 233 of the OPD compensator 231 to the peripheral region 232 can be 0.1 mm all the way to 1 mm, in some embodiments. It is possible that the image quality may start to suffer at an upper limit of the OPD. In order to get the largest OPD and minimize the aberration, it is advantageous for the OPD compensator 231 to use optical materials of high index of refraction for the OPD compensator 231. However, the OPD compensator 231 can also include optical materials of low index of refraction and include a complex aspherical surface. The OPD compensator 231 can also use materials with gradient index profile, either along the optical axis or radial direction. The OPD compensator 231 can include a spherical or an aspherical surface. The radius of curvature for the spherical surface of the OPD compensator 231 could be between 200 mm to 20 mm. Even the aspherical surface, the radius for the base curvature is also within the range. The aperture of the OPD compensator 231 can be between the 10 mm to 30 mm.

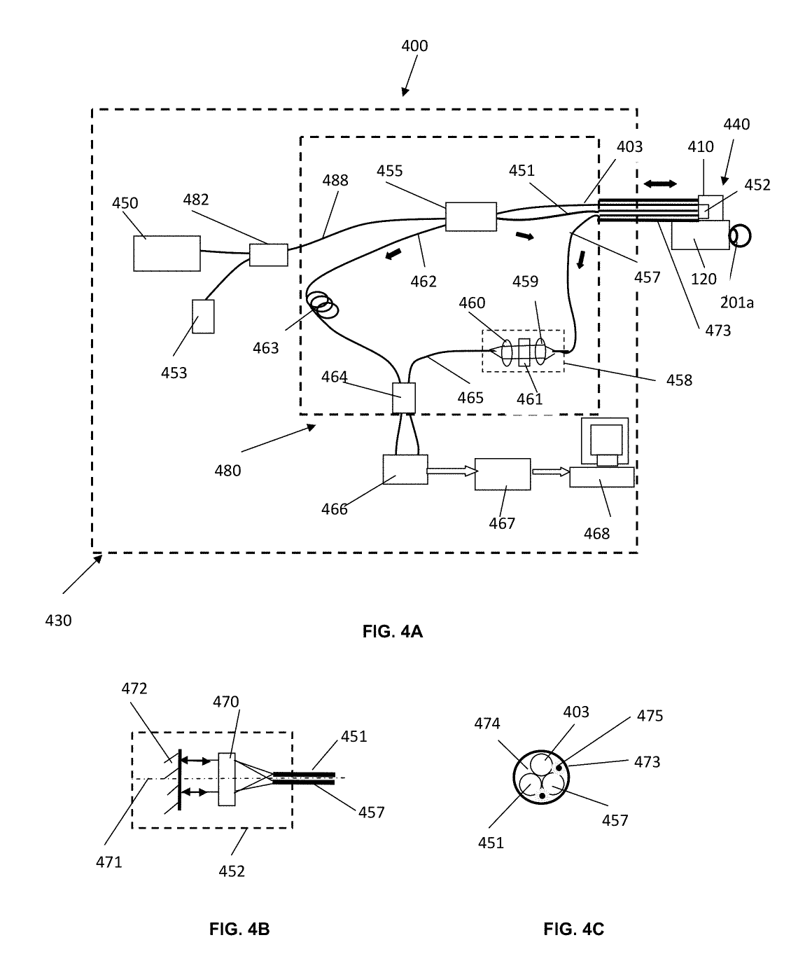

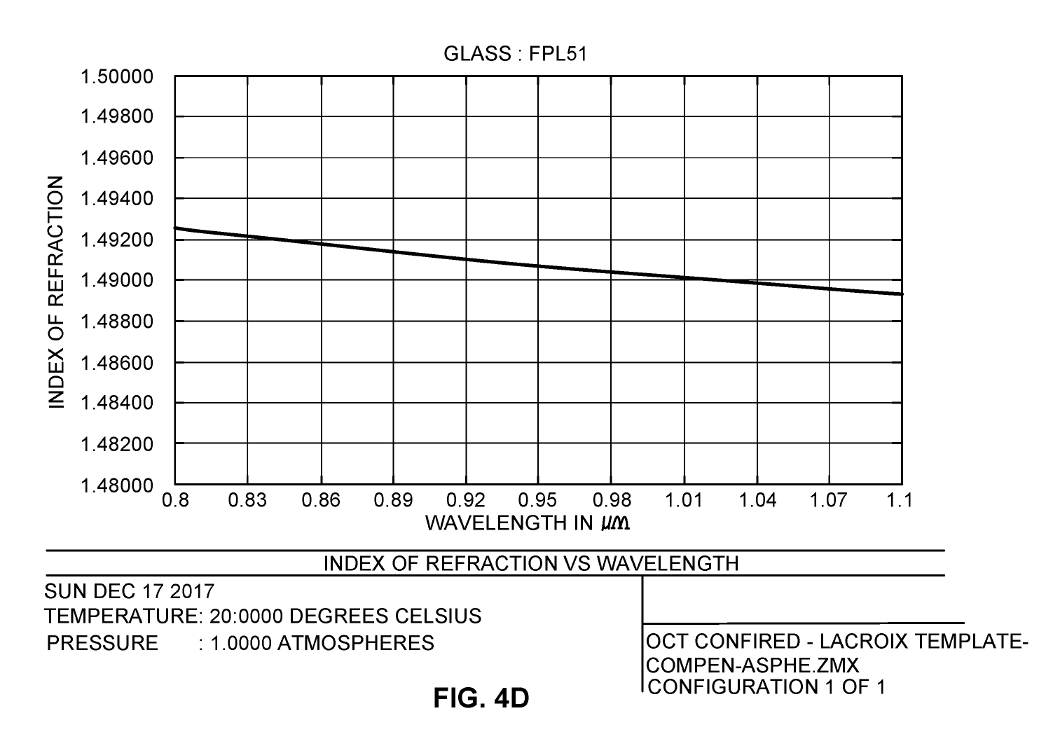

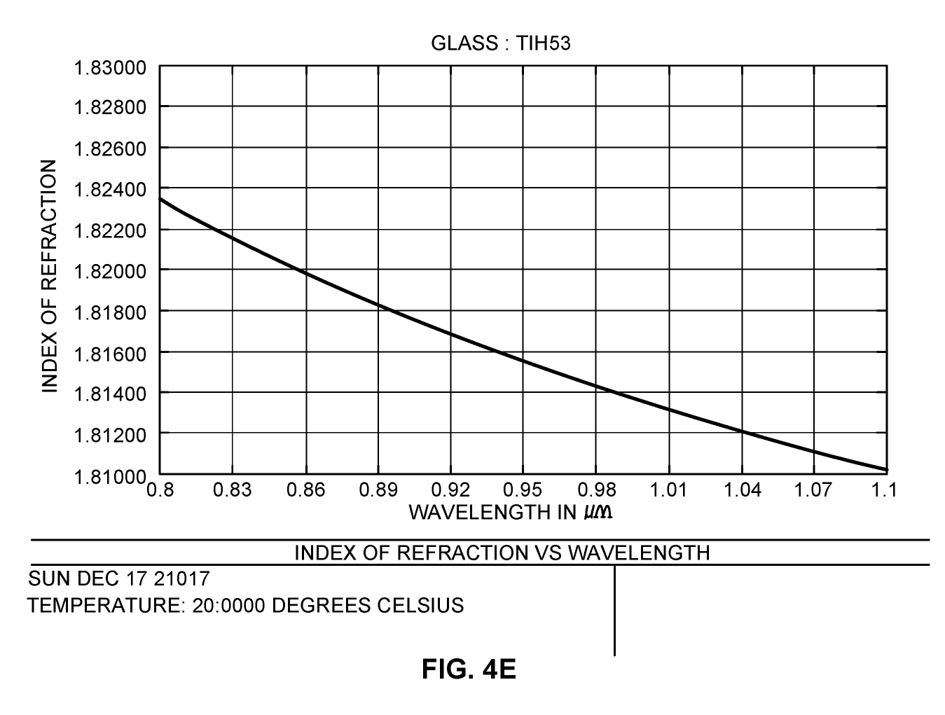

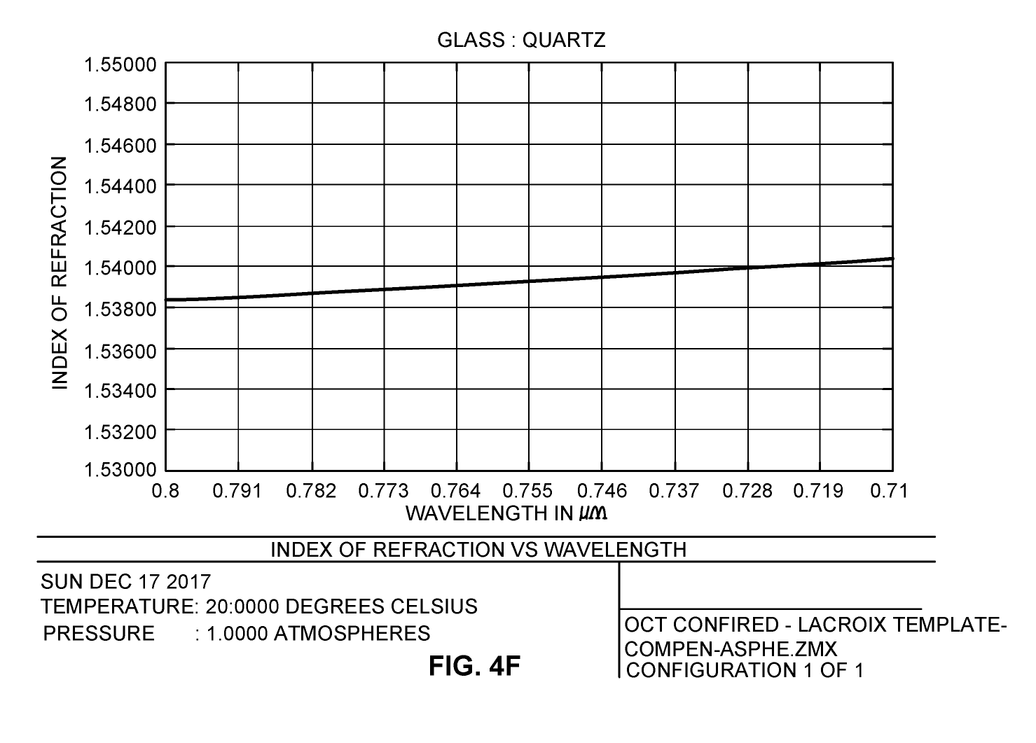

FIG. 4A illustrates a block diagram of an ultra-wide FOV OCT imaging system 400 with an imaging probe 440 and an interferometer 480 according to some embodiments of the disclosure. The ultra-wide FOV OCT imaging system 400 can include the imaging probe 440, a console 430 and a cable 473. The imaging probe 440 can include an optical window (not shown), a first imaging module 120, and a second imaging module 410. The optical window configured to be in contact with a cornea of an eye 201a. The first imaging module 120 includes a first light source (not shown) and configured to direct a first light beam of the first light source through the optical window to the eye and to form a first image of the eye. The second imaging module 410 is configured to receive a second light beam from a second light source 450 and to direct the second light beam through the optical window to the eye and to receive reflected light of the second light beam from the eye. The console 430 can include the second light source 450, an interferometer 480, a balanced optical detector 466, a data acquisition module 467, and a processor (with a display) 468. The interferometer 480 is configured to receive the reflected light from the second imaging module 410 and to generate data of the interference. The processor 468 is configured to process the data from the interferometer 480 and to generate the second image, which is an OCT image.

The ultra-wide FOV OCT imaging system 400 can further include the cable 473 coupled between the console 430 and the imaging probe 440. The cable 473 can include a first fiber 403 and a second fiber 451. The first fiber 403 is configured to transmit a sample arm of the second light beam from the second light source 450 to the second imaging module 410 and to transmit reflected light of the sample arm from the second imaging module 410 to the interferometer 480. In some embodiments, the second fiber 451 is configured to transmit a reference arm of the second light beam from the second light source 450 to the second imaging module 410 and to transmit reflected light of the reference arm from the second imaging module 410 to the interferometer 480.

In some other embodiments, the cable 473 can further include a third fiber 457, where the second fiber 451 is configured to transmit a reference arm of the second light beam from the second light source 450 to the second imaging module 410, and the third fiber 457 is configured to transmit reflected light of the reference arm from the second imaging module 410 to the interferometer 480, as shown in FIG. 4A. For the reference beam, the reflection module 252 in the FIG. 2A is replaced by a reflection module 452 for the embodiments in FIG. 4A. Instead of using of a dual fiber configuration, the new configuration is implemented with a triple fiber (fiber 403, 451 and 457) construction. The reference light beam is reflected back to the OCT interferometer 480 through the fiber 457, and then guided to a dispersion compensation module 458, then eventually to a balanced detector 466, in some embodiments.

The reflection module 452, shown in FIG. 4B, is similar in construction from the reflection module 252 shown in FIG. 2B, but different in the working principle because two fibers are used in the reflection module 452. In the reflection module 452, although a collimating lens 470 is placed with a back focusing plane located at or near an end of the fiber 451 and with an optical axis 471 parallel to the axis of the fiber 451, a slight shift of fiber 451 from the optical axis 471 of lens 470 is required. The end of fiber 457 should be in the focal plane of the collimating lens 470 while the fiber 457 is located in the opposite of the optical axis 471 of the lens 470 from the fiber 451 but with an equal distance from the optical axis 471. Therefore, the light beam exiting from the fiber 451 would form a collimated, but slightly tilted, light beam to illuminate a reflective surface 472. The reflector (mirror) 472 is placed to be perpendicular to the optical axis 471 of the collimating lens 470. The light reflected by the reflective surface 472 is then re-focused by the collimating lens 470, along slightly different optical path, to the end of the fiber 457 and return to the optical fiber 457 in opposite transmission direction.

The cable 473 shown in FIG. 4C is formed with optical fiber 403, 451 and 457 inside to make three fibers experiencing same or very similar deformation when the cable is stretched, twisted or bended during the use of the OCT imaging probe. In the embodiment, the three fibers, 403, 451 and 457 are placed within the cable sheath 473 to form a more stable triangle style of construction, which is easier to construct. To protect the optical fibers, a sheath can be added and be made of strong material to take most of the load when the cable 473 is stepped on while still flexible enough for the cable 473 when it is bent. Cable filling compound 474 can be made of jelly type of material to fill the space between the optical fibers and the sheath. Additional strength member or ripcord 475 can be placed within the cable sheath to enhance the stretching strength of the cable 473 and to reduce the direct stretching on the fibers.

In some embodiments, the second light source 450 is a swept light source. The light beam from the swept light source 450, which is linearly polarized, is guided through an optical fiber to an optical fiber combiner 482. A light beam from an aiming light source 453 is also guided to the combiner 482. The light beam from the swept light source 450 is coupled with the light beam from the aiming light source 453 at the combiner 482. The output light beam from the combiner 482 is guided by a fiber 488. For example, the fiber 488 can be a polarization maintaining (PM) fiber 488. The fiber 488 can guide output light beam from the combiner 482 into a port of PM fiber optic coupler 455 with polarization of the light beam aligned with one of polarization axes of fiber 488 (slow axis, for example), and then split into two light beams with various intensity ratios, (for example, ratios of 10:90, 20:80, or 30:70, or other ratios), which are then guided out through two PM optical fibers 403 and 451. A first portion of the output beam of the fiber optical coupler 455 can be guided to the PM fiber 403 to form the sample arm of the OCT interferometer 480, and a second portion of the output beam of the fiber optical coupler 555 can be guided to the PM fiber 451 to form the reference arm of the OCT interferometer 480. The light in the fiber 403 can be then further guided to the OCT imaging module 410 to form the sample arm of the optical fiber interferometer 480 in some embodiments. In some embodiments, the details of the sample arm in the second imaging module 410 can be as shown in FIG. 2A, or in FIG. 3B.

The light beam in the fiber 451 can be used to form the reference arm of the OCT interferometer 480, and is guided to the second imaging module (OCT imaging module) 410. As shown in FIG. 4A, the light in the fiber 451 is reflected backward by the reflection module 452 in the second imaging module 410 and returns to interferometer 480 through the fiber 457. The returned light in fiber 457 then is guided to the optical dispersion compensation module 458.

Disclosed herein is also the ultra-wide field of view (FOV) optical coherence tomography (OCT) imaging system 400 including the dispersion compensation module 458 according to some embodiments. The dispersion compensation module 458 can be disposed in an optical path of the reference arm portion and configured to perform dispersion compensation of the OCT imaging system. The optical dispersion compensation module 458 can include a collimator lens 459, an optical dispersion component 461 and another lens 460. Here the light beam from the fiber 457 is collimated by the collimator lens 459 and then coupled into another optical fiber 465 by a similar optical lens 460. The distance between the two optical lenses 459 and 460 can be changed to adjust the overall optical path length of the reference arm. In optics, dispersion is the phenomenon in which the phase velocity of a wave depends on its frequency. The optical dispersion component 461 can include various optical materials matching optical dispersion properties of the optical materials used to make the optics in the sample arm. The optical dispersion component 461 can also include an optical filter which absorbs or reflects the light beam from the aiming light source 453 and prevent the aiming light beam from going through the dispersion compensation module 458.

The second imaging module 410, the OCT imaging module, can include a plurality of optical lenses, for example, the lenses 104, 114, 202, 204 and 205, as shown in FIG. 2A or FIG. 3B. The plurality of optical lenses can be configured for full FOV dispersion compensation. The plurality of optical lenses can include a plurality of optical materials. The plurality of optical materials can be selected such that a difference between a first total dispersion of a first optical path length of the sample arm portion in the center and a second total dispersion of a second optical path length of the sample arm portion along the peripheral region of a full FOV is reduced, thereby an axial resolution of a second image is improved.

The total dispersion is the dispersion between two extreme wavelengths, a longest wavelength and a shortest wavelength in a wavelength range of the second light source. In some embodiments, the plurality of optical lenses are configured to reduce an optical path difference (OPD) between the longest wavelength and the shortest wavelength in the wavelength range of the second light source to be a constant for the full FOV of the OCT imaging system 400 by selecting the plurality of optical materials. In some embodiments, the dispersion compensation module 458 is configured to compensate a residual OPD for the full FOV of the OCT imaging system 400. The residual OPD is the remaining OPD between the longest wavelength and the shortest wavelength in the wavelength range of the second light source for the full FOV after OPD being reduced or compensated by the plurality of optical lenses through selecting the plurality of optical materials.

A method to perform full field dispersion compensation is also disclosed herein. In some embodiments, the OCT imaging system 400 can have dispersion compensation for the full FOV. In the ultra-wide FOV OCT imaging system 400, the wavelength of the light source is either spread in certainly range, for example, 50 nm or even more than 100 nm, or scanned rapidly from one short wavelength to a longer wavelength with range of 50 nm or even more than 100 nm, during the data acquisition of a A-scan. For the ultra-wide FOV OCT imaging system 400, the issue of dispersion compensation within an OCT image starts to show its effect. The concept of such kind of dispersion has an analogy to chromatic aberration correction in the design of a typical optical color imaging system. FIGS. 4D-4F illustrate three dispersion plots of three different optical materials, which illustrate different index of refraction at different wavelengths. At the same span of wavelength, not only the three different optical materials have different index of refraction, but also the slops of the curve or the changes of the index of the refraction are different. For most of optical materials, the index of refraction is reduced as the wavelength becomes longer. For quartz, at certain condition, or certainly type of polycarbonate, the index of refraction actually increases.

By selection different optical materials with different dispersion characteristic to make the imaging lenses, where some of the imaging lenses are positive lenses and some are negative lenses, the total dispersion for the light rays located in the center or the peripheral region of the full field, which not only depends on the dispersion characteristic but also the optical path within the lenses for each light ray, could be reduced to the level that does not affect the axial resolution of the OCT image. In some embodiments, the optical dispersion of the light rays in the full FOV, resulted from the range of scanning wavelengths, can be maintained as a constant as possible, through the selection of optical materials and optimization of the optical design. The full field dispersion compensation method is advantageous over digital or software dispersion compensation algorithm to achieve the dispersion compensation because digital or software dispersion compensation algorithm will inevitably introduce the artifacts in the resulting OCT images.

The full field dispersion compensation is configured to make the OPD or group velocity at the two extreme wavelength of the OCT light source for each of the light beams to be close to a constant or ideally to be zero, where the light beams can pass along a center or a peripheral region of the object. If the OPD for dispersion is made to be a constant for the full field, then the residual OPD for each of the light beams can be compensated by the dispersion compensation element 461 in the reference beam. If the residual OPD is significantly different for the light beams from the center to the peripheral region of the object, the compensation element 461 in the reference beam can only get the dispersion corrected for certain regions of the OCT image, but not for the full field. A digital dispersion compensation algorithm could be used to get the dispersion corrected, for example, different light beams in the different parts of OCT image could use different dispersion compensation parameters. However, the digital dispersion compensation algorithm is more computation extensive, since the OPD for two extreme wavelength of the light source for each of light beams is in the order of about 4-12 microns. Therefore, the full field dispersion compensation is advantageous to obtain a sharp OCT image with high axial resolution from the center to the peripheral region of the object.

Referring to FIG. 4A, the light beam scattered form the retina 201 of the eye can be received by the OCT imaging module 410 and sent back through the optical fiber 403. The light beam is then returned to the fiber optic interferometer 480 through the optical fiber 403. Although the returned light beam comprising light beams originated from the swept light source 450 and the aiming light source 453, the intensity of the aiming light is more attenuated because the fact that the beam splitting dichroic mirror within the OCT Imaging Module 410 transmits a significant portion of the light beam of the aiming light source 453 to the image sensor.

A portion of the light beam from the fiber 403 can be split by the optical fiber coupler 455 again, which is then coupled to another fiber 462. A polarization adjustment component 463 can be used to adjust the polarization direction of the light transmitting from the fiber 462 to another 50:50 optical fiber coupler 464, and to align with the polarization direction of light beam from the fiber 465 in the reference arm. The light beams from the sample arm fiber 462 and the reference arm fiber 465 can be mixed in the fiber coupler 464, resulting in two interfering light beams with a constant 180 degree shift in their optical phase difference, which are then coupled out in two optical fibers. The balance optical detector 466 can be used to detect the light signals from the two fibers and converts them into electrical signal in the form of differential detection. The digital acquisition module 467 can be used to digitize the analog signal which is then sent to the processor 468 for further processing and for generating OCT images for physicians to make medical diagnosis. The processor 468 is configured to process data from the interferometer 480 and to generate the OCT image of the eye from the OCT imaging module 410 in the case of ophthalmic applications and OCT image of other objects in the case of other applications. Because most of the light beam from the aiming light source 453 is removed before reaching the balance detector 466, it cannot cause any significant effect in the detected signals. In some other embodiments, the balance detector 466 can be configured such that the detector 466 is not sensitive to the light wavelength range of the aiming light source 453.

For conventional OCT imaging systems where the optical fibers between the OCT engine and the scanning probe are relatively short and fixed in space, the motion effect, such as bending or twisting of the fibers, may not be significant on those fibers during the operation of OCT imaging. However, in order to be connected to a portable handheld imaging probe or a remote imaging probe 440, the fibers can be long, for example, as long as 5 meters. For the portable handheld OCT imaging probe 440, because the fibers are long and being part of the optical interferometer, such motion effect on the fibers, for example, bending or twisting of fibers, can introduced unwanted instability of the OCT images and affect the image quality significantly. One of the most significant problems for the conventional handheld OCT imaging probes is such motion effect.

As shown in FIG. 4A, the use of the triple fiber configuration (e.g., the fibers 403, 451 and 457), and the related optical design for the OCT interferometer 480 can reduce and minimize such motion effect. Because the three fibers 403, 451 and 457 are closely fixed inside one single long cable 473, the external effects, such as motion effect from stretching, bending or twisting of the fibers 403, 451 and 457, would change the polarization direction as well as optical path of light beam transmitting in the fibers in same fashion and same amount. However, such motion induced optical effect could be cancelled out or minimized by the optical interferometer 480. Therefore, the resulted OCT images are more stable. The stabilization of the polarization direction for the light beam in the sample arm is advantageous in stabilizing the optical interference pattern in the fiber interferometer 480, which can result in more stable OCT images and increase the image quality significantly.

In some other embodiments, an optical coherence tomography (OCT) imaging system can only include an imaging probe, where a dispersion compensation module is disposed in the imaging probe. The handheld imaging probe can comprise an optical window, a first imaging module (for example, a color imaging module), a second imaging module (an OCT imaging module) and a console. The console can be disclosed inside the imaging probe. The console can be a miniature console integrated with the imaging probe. The console can comprise an OCT engine and a scanning mirror controller. The OCT engine can comprise an interferometer, a second light source (for example, an OCT light source with wavelength from 800 nm to 1100 nm), a light detector, a data acquisition system, and a processor. The console can be connected to the second imaging module by one or more fibers disposed inside the imaging probe. A second light beam of the second light source can have a sample arm beam portion and a reference arm beam portion. The imaging probe can further include the dispersion compensation module disposed in an optical path of the reference beam portion in the console, where the console is inside the imaging probe.

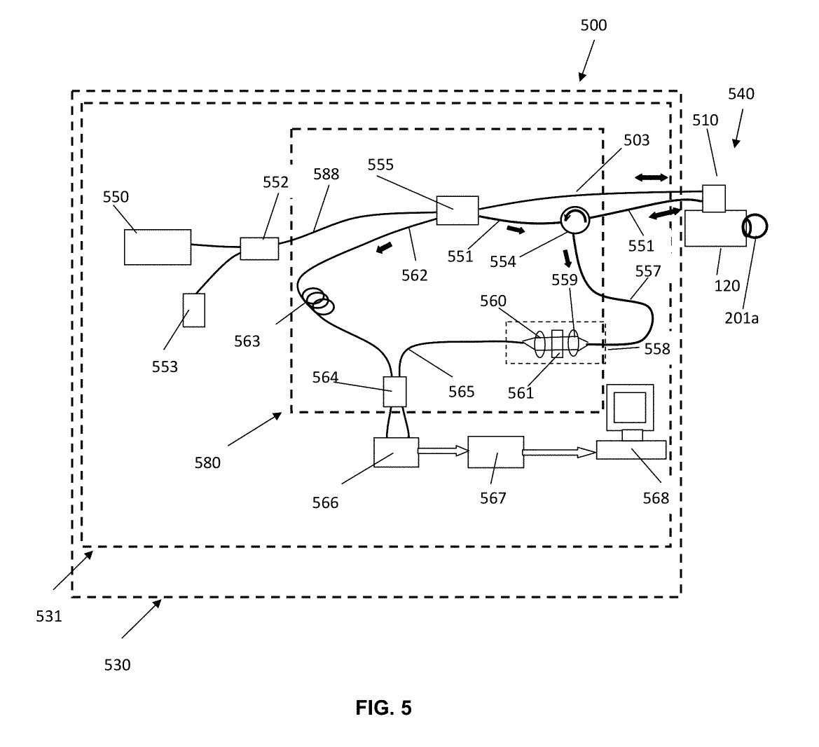

FIG. 5 is a block diagram of an OCT imaging system 500 including an optical circulator 554 in a reference arm portion according to some embodiments of the disclosure. The OCT system 500 can include an optical window, a first imaging module 120 and a second imaging module 510. The optical window is disposed at a distal end of the imaging probe 540 and configured to be in contact with a cornea of an eye 201a. The first imaging module 120 is disposed inside the imaging probe 540 and configured to form a first image of the eye 201a. The first imaging module 120 can include a first light source and a light conditioning element. The first light source is configured to provide a first light beam. The light conditioning element is configured to direct the first light beam through the optical window to the eye 201a. The second imaging module 510 is disposed inside the imaging probe 540 and configured to receive a second light beam from a second light source 550. The second light source 550 is configured to provide the second light beam. The second light beam includes a sample arm portion and a reference arm portion. The OCT system 500 can further include a beam splitter disposed inside the imaging probe 540 and configured to transmit the first light beam and to reflect the sample arm portion of the second light beam. The OCT system 500 can further include the optical circulator 554 disposed in an optical path of a light beam portion. The light beam portion includes at least one of the sample arm portion or the reference arm portion. The optical circulator 554 includes a first port, a second port, and a third port. The first port is configured to receive the light beam portion and to transmit the light beam portion to the second port. The second port is configured to transmit the light beam portion out, for example, to a second fiber 551, and to receive retuned light of the light beam portion and to transmit the retuned light of the light beam portion to the third port.

The OCT imaging system 500 can comprise the imaging probe 540 and a console 530. The console 530 can comprise an OCT engine 531 and a scanning mirror control (not shown). The OCT engine 531 can comprise an OCT interferometer 580, a second light source (an OCT light source) 550, a balanced optical detector 566, a data acquisition module 567, a processor (with a display) 568. For example, the OCT interferometer 580 can be a fiber optical interferometer. The OCT light source 550 can comprise a swept light source in some embodiments. The OCT interferometer 580 can work with different handheld imaging probes with different OCT imaging modules. For example, the OCT interferometer 580 can work with the OCT imaging module 210 in FIG. 2A, an OCT imaging module 310 in FIG. 3, and a variety of other OCT imaging modules.

In some embodiments, the light beam from the swept light source 550, which is linearly polarized, is guided through an optical fiber to an optical fiber combiner 552. A light beam from an aiming light source 553 is also guided to the combiner 552. The light beam from the swept light source 550 is coupled with the light beam from the aiming light source 553 at the combiner 552. The output light beam from the combiner 552 is guided by a fiber 588. For example, the fiber 588 can be a polarization maintaining (PM) fiber 588. The fiber 588 can guide output light beam from the combiner 552 into a port of PM fiber optic coupler 555 with polarization of the light beam aligned with one of polarization axes of fiber 588 (slow axis for example), and then split into two light beams with various intensity ratios, (for example, ratios of 10:90, 20:80, or 30:70, or other ratios), which are then guided out through two PM optical fibers 503 and 554. A first portion of the output beam of the fiber optical coupler 555 can be guided to the PM fiber 503 to form a sample arm of the OCT interferometer 580, and a second portion of the output beam of the fiber optical coupler 555 can be guided to the PM fiber 551 to form a reference arm of the OCT interferometer 580.

Referring to FIG. 5, the light in the fiber 503 can be then further guided to the OCT imaging module 510 to form the sample arm of the optical fiber interferometer 580 in some embodiments. In some embodiments, the details of the sample arm in the second imaging module 510 are shown in FIG. 2A, or in FIG. 3B.

The light beam in the fiber 551 can be used to form the reference arm of the OCT interferometer 580, after passing through an optical circulator 554, and is guided to the OCT imaging module 510. As shown in FIG. 5, the light in the fiber 551 is reflected backward by a reflection module (not shown in FIG. 5, similar to 252 in FIG. 2A) and returns to interferometer 580 through the same optical fiber 551. The returned light in fiber 551 then is guided to the port of the optical circular 554 which is connected to optical fiber 557. The optical circulator is an optical device with three ports, in which the light entering from one designated port will exit in a second designated port, while the light returned to the second port will exit from third port with very small amount of light returning to first port due to the use of an optical isolator.

The light beams exiting the optical circulator in the fiber 557 is further guided to an optical dispersion compensation module 558. Here the light beam from the fiber 557 is collimated by a collimator lens 559 and then coupled into another optical fiber 565 by a similar optical lens 560. The distance between the two optical lenses 559 and 560 can be changed to adjust the overall optical path length of the reference arm. The optical dispersion component 561 can comprise various optical materials matching optical dispersion properties of the optical materials used to make the optics in the sample arm. The optical dispersion component 561 can also include an optical filter which absorbs or reflects the light beam from the aiming light source 553 and prevent the aiming light beam from going through the dispersion compensation module 558.

Referring to FIG. 5, the light beam scattered form the retina of the eye 201a can be received by the OCT imaging module 510 and sent back through the optical fiber 503. The light beam is then returned to the fiber optic interferometer 580 through the optical fiber 503. Although the returned light beam comprising light beams originated from the swept light source 550 and the aiming light source 553, the intensity of the aiming light is more attenuated because the fact that the beam splitting dichroic mirror within the OCT imaging module 510 transmits a significant portion of the light beam of the aiming light source 553 to the image sensor.

A portion of the light beam from the fiber 503 can be split by the optical fiber coupler 555 again, which is then coupled to another fiber 562. A polarization adjustment component 563 can be used to adjust the polarization direction of the light transmitting from the fiber 562 to another 50:50 optical fiber coupler 564, and to align with the polarization direction of light beam from the fiber 565 in the reference arm. The light beams from the sample arm fiber 562 and the reference arm fiber 565 can be mixed in the fiber coupler 564, and results in two interfering light beams with a constant 180 degree shift in their optical phase difference, which are then coupled out in two optical fibers. The balance optical detector 566 can be used to detect the light signals from the two fibers and converts them into electrical signal in the form of differential detection. The digital acquisition module 567 can be used to digitize the analog signal which is then sent to the processor 568 for further processing and for generating OCT images for physicians to make medical diagnosis. The processor 568 is configured to process data from the interferometer 580 and to generate the OCT image of the eye from the OCT imaging module 510 in the case of ophthalmic applications and OCT image of other objects in the case of other applications. Because most of the light beam from the aiming light source 553 is removed before reaching the balance detector 566, it cannot cause any significant effect in the detected signals. In some other embodiments, the balance detector 566 can be configured such that the detector 566 is not sensitive to the light wavelength range of the aiming light source 553.

For conventional OCT imaging systems where the optical fibers between the OCT engine and the scanning probe are relatively short and fixed in space, the motion effect, such as bending or twisting of the fibers, may not be significant on those fibers during the operation of OCT imaging. However, in order to be connected to a portable handheld imaging probe or a remote imaging probe, the fibers can be long, for example, as long as 5 meters. For the portable handheld OCT imaging probe, because the fibers are long and being part of the optical interferometer, such motion effect on the fibers, for example, bending or twisting of fibers, can introduced unwanted instability of the OCT images and affect the image quality significantly. One of the most significant problems for the conventional handheld OCT imaging probes is such motion effect.

As shown in FIG. 5, the use of dual fiber configuration (e.g., fibers 503 and 551), and the related optical design for the OCT interferometer 580 can reduce and minimize such motion effect. As shown in FIG. 5, because two fibers are closely fixed inside one single long cable, the external effects, such as motion effect from stretching, bending or twisting of the fibers 503 and 551, would change the polarization direction as well as optical path of light beam transmitting in the fibers in same fashion and same amount. However, such motion induced optical effect could be cancelled out or minimized by the optical interferometer 580. Therefore, the resulted OCT images are more stable. The stabilization of the polarization direction for the light beam in the sample arm is very important in stabilizing the optical interference pattern in the fiber interferometer 580, which can result in more stable OCT images and increase the image quality significantly.

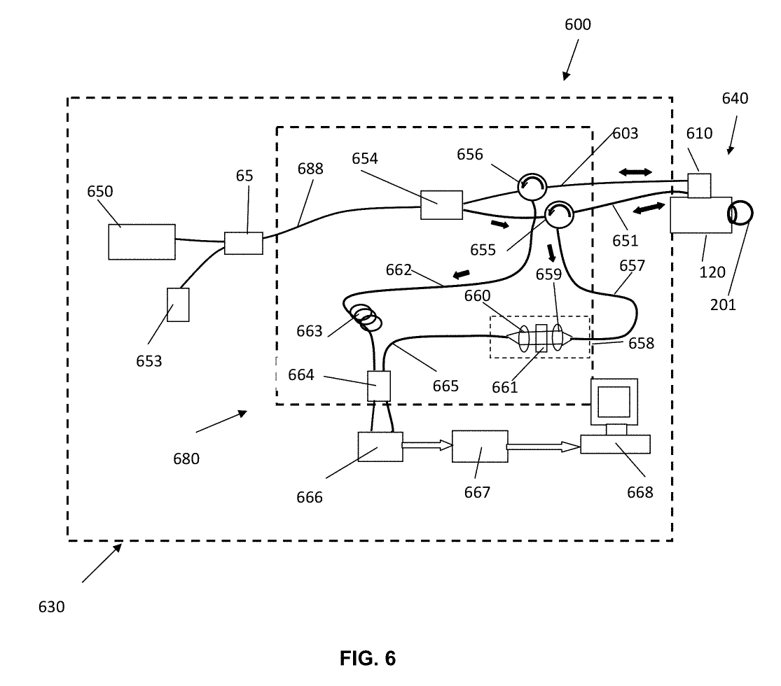

FIG. 6 illustrates an OCT imaging system 600 with an imaging probe 640 and an interferometer 680 according to another embodiment of the disclosure. Reference numbers in FIG. 6 are increased by 100 for similar components in FIG. 5. Here, an optical circulator 656 is added to the sample beam of interferometer 680 by splicing it into the optical fiber 603. The returned sample beam light from remote imaging module 610 is guided to fiber 662. A polarization adjustment component 663 can be used to adjust the polarization direction of the light transmitting from the fiber 662 to another 50:50 optical fiber coupler 664, and to align with the polarization direction of light beam from the fiber 665 in the reference arm. The light beams from the sample arm fiber 662 and the reference arm fiber 665 can be mixed in the fiber coupler 654, and results in two interfering light beams with a constant 180 degree shift in their optical phase difference, which are then coupled out in two optical fibers. Such configuration is advantageous to provide higher signal strength from the sample beam due to the elimination of light loss from the optic coupler 654.

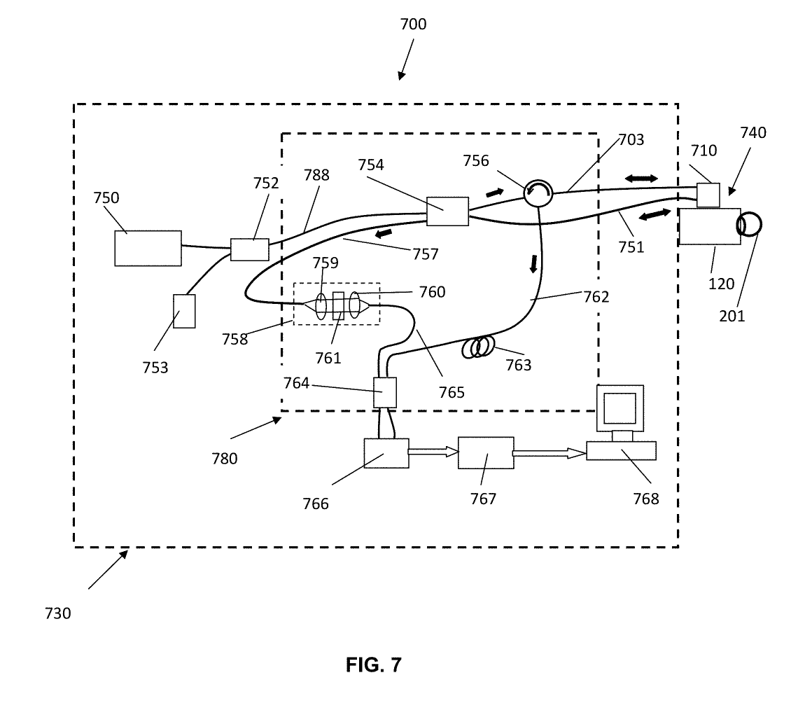

FIG. 7 illustrates an OCT imaging system 700 with an imaging probe 740 and an interferometer 780 according to yet another embodiment of the disclosure. Reference numbers in FIG. 7 are increased by 100 for similar components in FIG. 6. Here, only an optical circulator 756 is kept in the sample beam of interferometer 780 by splicing it into the optical fiber 703. The returned reference beam light from remote imaging module 710 is guided to optical fiber coupler 754, and then exit from port to fiber 757, which is further guided to an optical dispersion compensation module 758. Here the light beam from the fiber 757 is collimated by a collimator lens 759 and then coupled into another optical fiber 765 by a similar optical lens 760. The distance between the two optical lenses 759 and 760 can be changed to adjust the overall optical path length of the reference arm. The optical dispersion component 761 can comprise various optical materials matching optical dispersion properties of the optical materials used to make the optics in the sample arm. The optical dispersion component 761 can also include an optical filter which absorbs or reflects the light beam from the aiming light source 753 and prevent the aiming light beam from going through the dispersion compensation module 758. A polarization adjustment component 763 can be used to adjust the polarization direction of the light transmitting from the fiber 762 to another 50:50 optical fiber coupler 764, and to align with the polarization direction of light beam from the fiber 765 in the reference arm. The light beams from the sample arm fiber 762 and the reference arm fiber 765 can be mixed in the fiber coupler 754, and results in two interfering light beams with a constant 180 degree shift in their optical phase difference, which are then coupled out in two optical fibers.

In some other embodiments, an optical coherence tomography (OCT) imaging system can only include an imaging probe, where an optical circulator can be disposed in the imaging probe. The handheld imaging probe can comprise an optical window, a first imaging module (for example, a color imaging module), a second imaging module (an OCT imaging module) and a console. The console can be disclosed inside the imaging probe. The console can be a miniature console integrated with the imaging probe. The console can comprise an OCT engine and a scanning mirror controller. The OCT engine can comprise an interferometer, a second light source (for example, an OCT light source with wavelength from 800 nm to 1100 nm), a light detector, a data acquisition system, and a processor. The console can be connected to the second imaging module by one or more fibers disposed inside the imaging probe. A second light beam of the second light source can have a sample arm beam portion and a reference arm beam portion. The imaging probe can further include the optical circulator disposed in at least one of an optical path of the reference beam portion or an optical path of the sample arm portion in the console, where the console is inside the imaging probe.

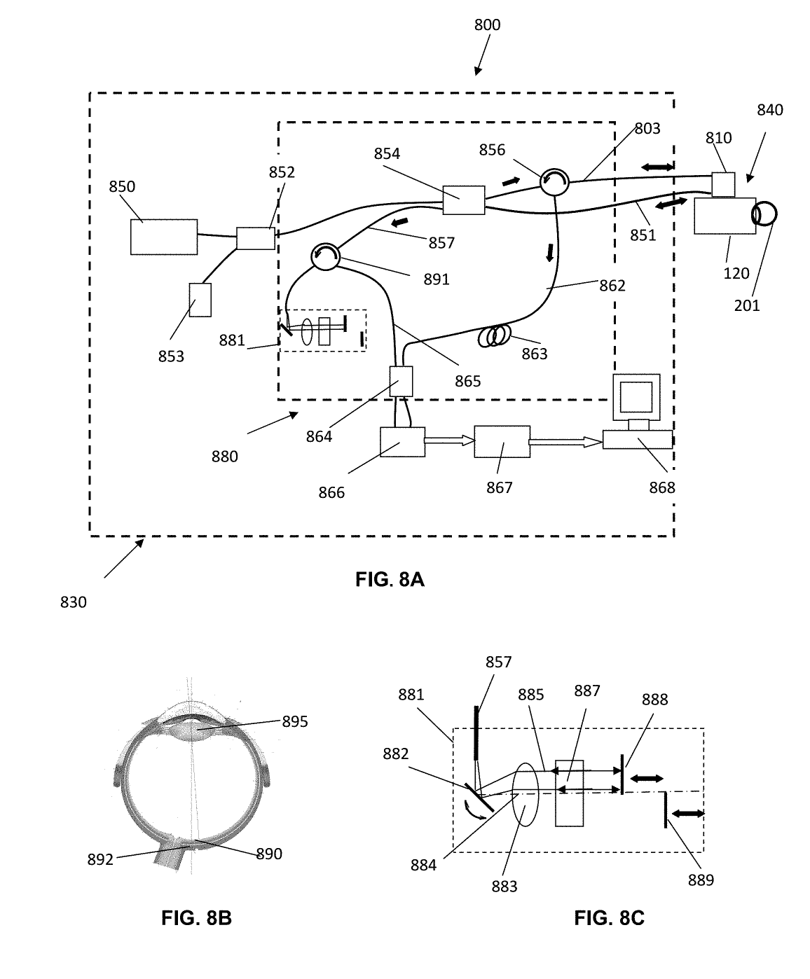

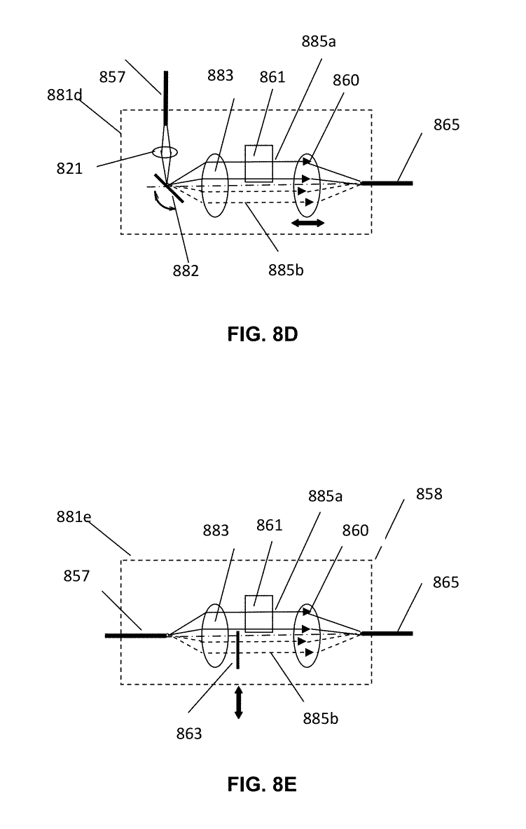

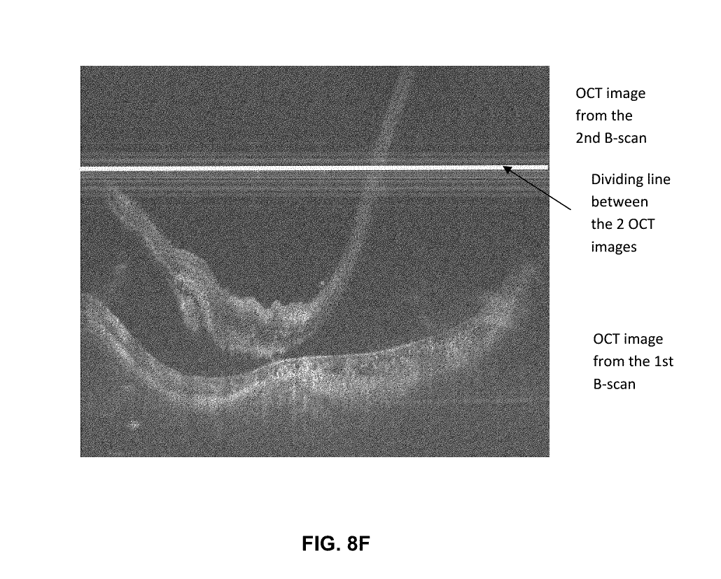

FIG. 8A is a block diagram of an OCT imaging system 800 including a two-channel OPD compensation unit 881 according to one embodiment of the disclosure. Reference numbers in FIG. 8 are increased by 100 for similar components in FIG. 7. Referring to FIG. 8A and FIG. 8C, in some embodiments, the OCT imaging system 800 can include the two-channel compensation unit 881 disposed in an optical path of the reference arm portion. The two-channel compensation unit 881 can include a first optical channel, a second optical channel, and an optical switching element 882. A first optical path of the first optical channel is shorter than a second optical path of the second optical channel. The optical switching element 882 is configured to switch the reference arm portion between the first optical channel and the second optical channel such that an axial optical depth of the OCT imaging system is doubled, thereby a field of view of the OCT imaging system is extended.

FIG. 8B illustrates an optical path difference between an anterior segment and a posterior segment of the eye. The OCT imaging technology inherently provides high axial resolution, however, the shortcoming is exhibited in the relatively short axial depth of the OCT imaging System. The axial depth of the OCT imaging system is also referred to as the image range or the axial depth of the OCT image. As shown in FIG. 8B, the OCT image can provide very detailed information about the retinal structure of the eye around surface of the retina 890. However, the structure of the tissue below retinal surface 890, such as an area in 892 cannot be seen in a single OCT scanned image. A quick second OCT scan to the area 892 immediately after first scan in area (depth) of surface 890 requires a very quick adjustment of the optical path length in the reference beam to reach a new balance in optical path length difference between the sample arm portion beam and the reference arm portion beam. In some instance, it is be valuable to provide an OCT image in other parts of the eye, for example, the posterior surface of the crystal lens 895 in the eye, quickly after a first scan of OCT image to the retinal surface 890 as well.

Referring back to FIG. 3A, where a folding in phenomena is exhibited when the axial optical depth of the object is larger than the axial optical depth of the OCT imaging system in the ultra-wide FOV OCT imaging system. An optically unfolding process can prove another solution to this problem. The optically unfolding process can be used to the axial optical depth of the OCT imaging system, thereby extending the field of view of the OCT imaging system. The optically unfolding process can be achieved by the two-channel OPD compensation unit 881.

Now referring to FIG. 8A, to achieve such fast adjustment of the optical path length in the reference arm portion, the two-channel OPD compensation unit 881 can in disposed in a reference arm portion of a second light beam of an OCT light source 850. The reference light beam in the fiber 857 is guided through an optical circulator 891 first, then to the compensation unit 881, which is a reflective type compensation module. When the conditioned reference light beam is returned to the optical circulator 891, it is guided to exit into the optical fiber 865, and then to the balanced detector 864.

FIG. 8C illustrates details of the two-channel OPD compensation unit in FIG. 8A. Referring to FIG. 8A and FIG. 8C, the two-channel OPD compensation unit 881 can include a first optical channel, a second optical channel, and an optical switching element 882, which is a fast rotating scan mirror 882. A first optical path of the first optical channel is shorter than a second optical path of the second optical channel. The optical switching element 882, the fast rotating scan mirror 882, is configured to switch the reference arm portion between the first optical channel and the second optical channel to adjust the optical path length in the reference arm portion. In this way, the achievable FOV of the OCT imaging system 800 can be extended.

A fast rotating scan mirror 882 is located between the fiber 857 and a collimating lens 883, with the rotating center laid on an optical axis 884 of the lens 883. The end of the fiber 857 is located along the extension of the optical axis 884 of the lens 883 when the bending of the axis by the scanning mirror 882 is considered, and at the focal point of the lens 883. As the result, the reference light beam 885 is collimated by the lens 883 within a scanning range (angle) of the scanning mirror 882, before striking on the reflector (mirror) 888 and 889. The optical dispersion component 887 can include various optical materials matching optical dispersion properties of the optical materials used to make the optics in the sample arm. The optical dispersion component 887 can also include an optical filter which absorbs or reflects the light beam from the aiming light source 853 and prevent the aiming light beam from going through the OPD compensation unit 881. The reflector 888 and 889 are placed perpendicular to the optical axis 884 of the lens 883, but at different distance from the lens 883, with both distances adjustable along the direction of the optical axis 884 of the lens 883. The reflector 888 and 889 are also placed on an opposite side of the optical axis 884 of the lens 883. The optical dispersion component 887 can have the same optical properties, including the overall optical path length along of the optical axis for light striking the reflector 588 and 589 in some embodiments. The optical dispersion component 887 can have different properties for optical path length for light striking the reflector 888 and 889 in some other embodiments.

As shown in FIG. 8C, when the fast scanning mirror 882 is set at an upper position shown, the light beam would be reflected back by the reflector 888, and eventually returned to the fiber 857. If the scanning mirror 882 is quickly switched to a lower position, the light beam would be reflected back by the reflector 889 with a different optical path length adjustment and eventually returned to fiber 857. If two OCT image scans are performed consequently, with the fast scanning mirror 882 switching between the two positions, the images will exhibit structures at two different areas of the eye. When the relative location of reflector 888 and 889 are adjusted carefully, it is possible to capture the structures in two adjacent areas with proper overlapping. Therefore, a composite OCT image can be generated to show effectively a larger imaging range (depth) than a single OCT image could show. The relative distance of the reflector 888 and 889 can also be adjusted such that one OCT image would show the retinal area while another one show the posterior of the crystal lens, in consecutive fashion.

Referring to FIG. 8C, the “reflective type” two-channel OPD compensation unit 881 not only be enable the ultra-wide field OCT imaging system 800 to image the anterior and posterior segment of eye, but also reduce the axial optical depth of the posterior segment, thereby extending the FOV of the OCT imaging system 800. Here the light from the reference beam fiber 857 is guided to the two-channel OPD compensation unit 881, where the light beam is focused by the focusing lens 883 to the scanning mirror 882, and becomes collimated by lens 883 which is placed with its back focal point located at a surface of the scanning mirror 882. Two optical paths are laid out with two mirrors 888 and 889 separately to reflect the light back to the lens 883, mirror 882, lens 883 and then the fiber 857. The location of the two mirrors 888 and 889 could be adjusted along the optical axis of lens 883 and set at different distance from lens 883 to create different optical path lengths. The mirror 882 could be driven to scan or switch between the two channels and result in different optical path lengths for returned reference beam portion, in which the optical path lengths are adjusted so that the axial optical depth of the object is slightly smaller than the axial optical depth of the OCT imaging system 800. As the result, there is a slight overlap between the two OCT images taken with the two reference arms generated by the OPD compensation unit 881. When such switching is synchronized with the B-scan frame of the OCT imaging system 800, two OCT images could be taken sequentially and at fast frame rate. The two OCT images then could be stitched together by software to display the OCT image with extended range.

FIG. 8D illustrates a two-channel OPD compensation unit 881d according to another embodiment of the disclosure. A “transmission type” of design is shown with the two-channel OPD compensation unit 881d which enables the function described in FIG. 8C. Here the light from the reference beam fiber 857 is guided to dual path OPD compensation unit 881d, where the light beam is focused by a focusing lens 821 to a scanning mirror 882, and become collimated by the lens 883 which is placed with its back focal point located at a surface of the scanning mirror 882. Two optical channels, 885a and 885b, are created along the two side of the optical axis of the lens 883. A second focus lens 860 is used to guide the collimated light beam into the optical fiber 865. An optical flat 861 made of transparent optical material is inserted into one of optical channels, which introduces a longer optical path for that channel due to the index of refraction from the material. The length of the optical flat is selected so that the increase in the optical path in channel 885a, compared to that in channel 885b, is slightly smaller than the axial optical depth of the OCT imaging system 800. The mirror 882 could be driven to scan or switch between the two channels and result in different optical path for the light received by the fiber 865.

FIG. 8E illustrates a two-channel OPD compensation unit 881e according to yet another embodiment of the disclosure. As shown in FIG. 8E, a shutter 863 is configured to be a switching element to switch the reference beam between the two optical channels 885a and 885b in order to adjust the optical path length of the reference arm portion of the second beam of the OCT light source. Instead of using the scanning mirror to steer the light beam, the shutter 863 is used to block the light in one of two channels at any given time, and only allows the light transmitted in one channel.

FIG. 8F illustrates an OCT image of a posterior segment of the eye, where a folding in phenomena is solved by optically unfolding. The optical unfolding can be achieved by OPD compensator and/or the two-channel OPD compensation unit in various embodiments. For example, the first channel of the two-channel OPD compensation unit can be configured to provide a first B-scan of a first OCT image, and the second channel of the two-channel OPD compensation unit can be configured to provide a second B-scan of a second OCT image. As shown in FIG. 8E, the first and the second OCT image can be combined. In this way, the axial optical depth of the OCT imaging system 800 can doubled, thereby the field of view of the OCT imaging system 800 can be significantly extended.

In some other embodiments, an optical coherence tomography (OCT) imaging system can only include an imaging probe, where the two-channel OPD compensation unit can be disposed in the imaging probe. The handheld imaging probe can comprise an optical window, a first imaging module (for example, a color imaging module), a second imaging module (an OCT imaging module) and a console. The console can be disclosed inside the imaging probe. The console can be a miniature console integrated with the imaging probe. The console can comprise an OCT engine and a scanning mirror controller. The OCT engine can comprise an interferometer, a second light source (for example, an OCT light source with wavelength from 800 nm to 1100 nm), a light detector, a data acquisition system, and a processor. The console can be connected to the second imaging module by one or more fibers disposed inside the imaging probe. A second light beam of the second light source can have a sample arm beam portion and a reference arm beam portion. The imaging probe can further include a two-channel OPD compensation disposed in an optical path of the reference beam portion in the console, where the console is inside the imaging probe.

In sum, an ultra-wide field Optical Coherence Tomography (OCT) imaging system with a remote imaging probe is disclosed. The ultra-wide FOV OCT imaging system can include an imaging probe. The imaging probe can include an optical widow configured to be in contact with a cornea of an eye. The imaging probe can include a first imaging module and a second imaging module. The first imaging module is configured to form a first image of the eye. For example, the first imaging module can be a two dimensional (2D) color imaging module. The first imaging module can include a first light source and a light conditioning element, where the first light source is configured to provide a first light beam and the light conditioning element is configured to direct the first light beam through the optical window to the eye. The second imaging module is configured to form a second image of the eye. For example, the second imaging module can be an OCT imaging module to provide a volumetric image of the eye. The second imaging module can include a scanning mirror configured to receive a sample arm portion of a second light beam from a second light source and scan the sample arm portion. The second light source can be disposed on a console, or in the imaging probe. The second light beam of the second light source can include the sample arm portion and a reference arm portion. The ultra-wide FOV OCT imaging system can further include a beam splitter configured to transmit the first light beam and to reflect the sample arm portion of the second light beam.

In some embodiments, the ultra-wide FOV OCT imaging system can further include a console and a cable coupled between the console and the imaging probe. The console can include the second light source, an interferometer, and a processor. The interferometer is configured to receive the reflected light from the second imaging module and to generate data of the interference between the sample arm portion and the reference arm portion of the second beam. The processor is configured to process the data from the interferometer and to generate the second image, which is the OCT image. The cable can include a first fiber and a second fiber, where the first fiber is configured to transmit the sample arm of the second light beam to the second imaging module in the imaging probe and to transmit reflected light of the sample arm from the second imaging module to the interferometer in the console; where the second fiber is configured to transmit a reference arm of the second light beam from the second light source to the second imaging module in the imaging module. In some embodiments, the cable further includes a third fiber, the third fiber being configured to transmit reflected light of the reference arm portion from the second imaging module to the interferometer. In some embodiments, the first fiber, the second fiber and the third fiber are closely fixed inside the cable such that external motion effects cause same changes in polarization direction and optical path lengths for the first fiber, the second fiber and the third fiber.

In some embodiments, the ultra-wide FOV OCT imaging system can include an optical path difference (OPD) compensator disposed in an optical path of the sample arm portion, the OPD compensator including a center and a peripheral region, wherein a first optical path along the center is shorter than a second optical path along the peripheral region such that an axial optical depth of the eye is reduced, thereby a FOV of the OCT imaging system is extended. In some embodiments, the OPD compensator is disposed closely to a secondary image plane of the second imaging module within 5 mm.

In some embodiments, the ultra-wide FOV OCT imaging system can include a dispersion compensation module. The dispersion compensation module can be disposed in an optical path of the reference arm portion and configured to perform dispersion compensation of the OCT imaging system. In some embodiments, the second imaging module, the OCT imaging module, can include a plurality of optical lenses, where the plurality of optical lenses are configured for full FOV dispersion compensation. The plurality of optical lenses can include a plurality of optical materials. The plurality of optical materials can be selected such that a difference between a first total dispersion of a first optical path length of the sample arm portion in the center and a second total dispersion of a second optical path length of the sample arm portion along the peripheral region of a full FOV is reduced, thereby an axial resolution of a second image is improved.

In some embodiments, the ultra-wide FOV OCT imaging system can include a two-channel optical path difference (OPD) compensation unit disposed in an optical path of the reference arm portion and including a first optical channel, a second optical channel, and an optical switching element, wherein a first optical path of the first optical channel is shorter than a second optical path of the second optical channel, wherein the optical switching element is configured to switch the reference arm portion between the first optical channel and the second optical channel such that an axial optical depth of the OCT imaging system is doubled, thereby a field of view of the OCT imaging system is extended.

In some embodiments, the ultra-wide FOV OCT imaging system can include an optical circulator disposed in an optical path of a light beam portion. The light beam portion includes at least one of the sample arm portion or the reference arm portion. The optical circulator includes a first port, a second port, and a third port. The first port is configured to receive the light beam portion and to transmit the light beam portion to the second port. The second port is configured to receive retuned light of the light beam portion and to transmit the retuned light of the light beam portion to the third port.

Advantageously, the OCT imaging system can have an ultra-wide field of view (FOV). Due to the contact nature of the OCT imaging module, a center of eye (more precisely, a center of the radius of curvature of the retina when being considered to be in a spherical shape) is used as a point to measure the FOV. In some embodiments, the FOV is 130 degrees×130 degrees and up to 180 degrees×180 degrees in a single volume acquisition. In terms of a scanning line on the retina, in some embodiments, the FOV of the OCT imaging system is about at least 20 mm×20 mm for new born infants, 24 mm×24 mm for children, and 28-30 mm×28-30 mm for adults, in a single volume acquisition. Because the periphery of the retina offers a unique perspective towards the assessment and monitoring of certain ocular diseases, the ultra-wide field of view of the OCT imaging system is valuable in detecting architectural changes caused by peripheral retinal pathology for the detection and diagnosis of various eye diseases.

In addition, the ultra-wide FOV OCT imaging system with an imaging probe is also advantageous to provide more stable OCT images and increase the image quality significantly. In some embodiments, the OCT imaging system includes a triple fiber configuration and the related optical design for the OCT interferometer. Because the three fibers are closely fixed inside one single long cable, the external effects, such as motion effect from stretching, bending or twisting of the fibers, would change the polarization direction as well as optical path of light beam transmitting in the fibers in same fashion and same amount. Thus, such motion induced optical effect could be cancelled out or minimized by the OCT interferometer. Therefore, the resulted OCT images are more stable with significantly increased image quality.

Furthermore, Moreover, the OCT imaging system is advantageous for infants, small children, bedridden patients, and animals because of easy alignment. The OCT imaging system can minimize the lateral alignment requirement between the eye of the user and the optical system of the OCT imaging system. Because the shape of the contact optical window is designed to fit closely with the shape of the cornea, it is easier to align the contact optical window with the cornea. The contact optical window can also reduce the free motion of the eye ball too. The OCT imaging system can provide high quality images for a group of users whose OCT images are difficult to be obtained by the conventional OCT imaging systems.

Various embodiments disclosed herein include:

### Embodiment 1

An ultra-wide field of view (FOV) optical coherence tomography (OCT) imaging system, comprising:

an imaging probe including an optical window, a first imaging module, and a second imaging module, the optical window configured to be in contact with a cornea of an eye, the first imaging module including a first light source and configured to direct a first light beam of the first light source through the optical window to the eye and to form a first image of the eye, the second imaging module configured to receive a second light beam from a second light source and to direct the second light beam through the optical window to the eye and to receive reflected light of the second light beam from the eye;

a console including the second light source, an interferometer configured to receive the reflected light from the second imaging module and to generate data, and a processor configured to process the data from the interferometer and to generate the second image; and

a cable coupled between the console and the imaging probe, the cable including a first fiber and a second fiber, the first fiber configured to transmit a sample arm portion of the second light beam from the second light source to the second imaging module and to transmit reflected light of the sample arm portion from the second imaging module to the interferometer, the second fiber configured to transmit a reference arm portion of the second light beam from the second light source to the second imaging module.

### Embodiment 2

The ultra-wide FOV OCT imaging system in Embodiment 1, wherein the cable further includes a third fiber, the third fiber being configured to transmit reflected light of the reference arm portion from the second imaging module to the interferometer.

### Embodiment 3

The ultra-wide FOV OCT imaging system in Embodiment 2, wherein the first fiber, the second fiber and the third fiber are closely fixed inside the cable such that external motion effects cause same changes in polarization direction and optical path lengths for the first fiber, the second fiber and the third fiber.

### Embodiment 4

The ultra-wide FOV OCT imaging system in Embodiment 2, wherein the first fiber, the second fiber and the third fiber are polarization maintaining fibers.

### Embodiment 5

The ultra-wide FOV OCT imaging system in Embodiment 1, wherein the imaging probe further includes a reflection module, the reflection module being configured to receive the reference arm portion of the second light beam from the second light source and to reflect back the reference arm portion to the interferometer.

### Embodiment 6

The ultra-wide FOV OCT imaging system in Embodiment 1, further comprising an aiming light through the first fiber into the second imaging module, wherein the first imaging module is configured to provide a registration for the second imaging module.

### Embodiment 7

The ultra-wide FOV OCT imaging system in Embodiment 1, wherein the first imaging module further includes a light conditioning element configured to directional control the first light beam to the eye, wherein the first imaging module has a field of view of 130 degrees.

### Embodiment 8

The ultra-wide FOV OCT imaging system in Embodiment 1, wherein the second imaging module further includes a MEM scanning mirror configured to receive the sample arm portion of the second light beam and scan the sample arm portion.

### Embodiment 9

The ultra-wide FOV OCT imaging system in Embodiment 1, wherein the second imaging module further includes a beam splitter configured to transmit the first light beam and to reflect the sample arm portion of the second light beam.

### Embodiment 10

The ultra-wide FOV OCT imaging system in Embodiment 1, wherein the second imaging module further includes an optical path difference (OPD) compensator disposed closely to a secondary image plane of the second imaging module within 1 mm, the OPD compensator including a center and a peripheral region, wherein a first optical path of the sample arm portion along the center is shorter than a second optical path of the sample arm portion along the peripheral region.

### Embodiment 11

The ultra-wide FOV OCT imaging system in Embodiment 1, further comprising a two-channel optical path difference (OPD) compensation unit disposed in an optical path of the reference arm portion and including a first optical channel, a second optical channel, and an optical switching element, wherein a first optical path of the first optical channel is shorter than a second optical path of the second optical channel, wherein the optical switching element is configured to switch the reference arm portion between the first optical channel and the second optical channel.

### Embodiment 12

The ultra-wide FOV OCT imaging system in Embodiment 1, further comprising a dispersion compensation module disposed in the reference arm portion and configured to perform dispersion compensation for a full field of view of the OCT imaging system such that an axial resolution of the OCT imaging system is improved.

### Embodiment 13

The ultra-wide FOV OCT imaging system in Embodiment 1, further comprising an optical circulator including three ports and disposed in at least one of an optical path the sample arm or an optical path of the reference arm.

### Embodiment 14

The ultra-wide FOV OCT imaging system in Embodiment 1, wherein a field of view of the OCT imaging system is 130 degrees by 130 degrees in a single volume acquisition.

### Embodiment 15

An ultra-wide field of view (FOV) optical coherence tomography (OCT) imaging system, comprising:

an imaging probe including
an optical window configured to be in contact with a cornea of an eye;
a first imaging module configured to form a first image of the eye, the first imaging module including a first light source and a light conditioning element, the first light source configured to provide a first light beam, the light conditioning element configured to direct the first light beam through the optical window to the eye;
a second imaging module configured to form a second image of the eye, the second imaging module including a scanning mirror configured to receive a sample arm portion of a second light beam from a second light source and scan the sample arm portion;

a beam splitter configured to transmit the first light beam and to reflect the sample arm portion of the second light beam; and

an optical path difference (OPD) compensator disposed in an optical path of the sample arm portion, the OPD compensator including a center and a peripheral region, wherein a first optical path of the sample arm portion along the center is shorter than a second optical path of the sample arm portion along the peripheral region such that an axial optical depth of the eye is reduced, thereby a FOV of the OCT imaging system is extended.

### Embodiment 16

The ultra-wide FOV OCT imaging system in Embodiment 15, wherein the OPD compensator is disposed closely to a secondary image plane of the second imaging module within 5 mm.

### Embodiment 17

The ultra-wide FOV OCT imaging system in Embodiment 15, wherein the optical path difference between the first optical path and the second optical path is between 0.5 mm to 3 mm.

### Embodiment 18

The ultra-wide FOV OCT imaging system in Embodiment 15, further comprising an aiming light through the first fiber into the second imaging module, wherein the first imaging module is configured to provide a registration for the second imaging module.

### Embodiment 19

The ultra-wide FOV OCT imaging system in Embodiment 15, wherein the first imaging module has a field of view of 130 degrees.

### Embodiment 20

The ultra-wide FOV OCT imaging system in Embodiment 15, wherein the second imaging module further includes a MEM scanning mirror configured to receive the sample arm portion of the second light beam and scan the sample arm portion.

### Embodiment 21

The ultra-wide FOV OCT imaging system in Embodiment 15, further comprising a console and a cable coupled between the console and the imaging probe.

### Embodiment 22

The ultra-wide FOV OCT imaging system in Embodiment 21, wherein the console includes the second light source, an interferometer, and a processor, wherein the cable including a first fiber, a second fiber, and a third fiber, the first fiber configured to transmit a sample arm portion of the second light beam from the second light source to the second imaging module and to transmit reflected light of the sample arm portion from the second imaging module to the interferometer, the second fiber configured to transmit a reference arm portion of the second light beam from the second light source to the second imaging module, the third fiber being configured to transmit reflected light of the reference arm portion from the second imaging module to the interferometer.

### Embodiment 23

The ultra-wide FOV OCT imaging system in Embodiment 15, further comprising a two-channel optical path difference (OPD) compensation unit disposed in an optical path of the reference arm portion and including a first optical channel, a second optical channel, and an optical switching element, wherein a first optical path of the first optical channel is shorter than a second optical path of the second optical channel, wherein the optical switching element is configured to switch the reference arm portion between the first optical channel and the second optical channel.

### Embodiment 24

The ultra-wide FOV OCT imaging system in Embodiment 15, further comprising a dispersion compensation module disposed in the reference arm portion and configured to perform dispersion compensation of the OCT imaging system.

### Embodiment 25

The ultra-wide FOV OCT imaging system in Embodiment 15, further comprising an optical circulator including three ports and disposed in at least one of an optical path the sample arm or an optical path of the reference arm.

### Embodiment 26

The ultra-wide FOV OCT imaging system in Embodiment 15, wherein a field of view of the OCT imaging system is 130 degrees by 130 degrees in a single volume acquisition.

### Embodiment 27

An ultra-wide field of view (FOV) optical coherence tomography (OCT) imaging system, comprising:

optical window disposed at a distal end of an imaging probe and configured to be in contact with a cornea of an eye;

a first imaging module disposed inside the imaging probe and configured to form a first image of the eye, the first imaging module including a first light source and a light conditioning element, the first light source configured to provide a first light beam, the light conditioning element configured to direct the first light beam through the optical window to the eye;

a second imaging module disposed inside the imaging probe and configured to receive a second light beam from a second light source, the second imaging module includes a plurality of optical lenses, the second light source being configured to provide the second light beam, the second light beam including a sample arm portion and a reference arm portion;

a beam splitter disposed inside the imaging probe and configured to transmit the first light beam and to reflect the sample arm portion of the second light beam; and

a dispersion compensation module disposed in an optical path of the reference arm portion and configured to perform dispersion compensation of the OCT imaging system;

wherein the plurality of optical lenses includes a plurality of optical materials such that a difference between a first total dispersion of a first optical path length of the sample arm portion in the center and a second total dispersion of a second optical path length of the sample arm portion along the peripheral region of a full FOV is reduced and an axial resolution of a second image is improved.

### Embodiment 28

The ultra-wide FOV OCT imaging system in Embodiment 27, wherein the plurality of optical materials are configured to reduce an optical path difference (OPD) between a longest wavelength and a shortest wavelength in a wavelength range of the second light source to be a constant for the full FOV of the OCT imaging system.

### Embodiment 29

The ultra-wide FOV OCT imaging system in Embodiment 27, wherein the dispersion compensation module is configured to compensate a residual OPD for the full FOV of the OCT imaging system.

### Embodiment 30

The ultra-wide FOV OCT imaging system in Embodiment 27, further comprising an aiming light through the first fiber into the second imaging module, wherein the first imaging module is configured to provide a registration for the second imaging module.

### Embodiment 31

The ultra-wide FOV OCT imaging system in Embodiment 27, wherein the first imaging module has a field of view of about 130 degrees.

### Embodiment 32

The ultra-wide FOV OCT imaging system in Embodiment 27, wherein the second imaging module further includes a MEM scanning mirror configured to receive the sample arm portion of the second light beam and scan the sample arm portion.

### Embodiment 33

The ultra-wide FOV OCT imaging system in Embodiment 27, further comprising a console and a cable coupled between the console and the imaging probe.

### Embodiment 34

The ultra-wide FOV OCT imaging system in Embodiment 33, wherein the console includes the second light source, an interferometer, and a processor, wherein the cable including a first fiber, a second fiber, and a third fiber, the first fiber configured to transmit a sample arm portion of the second light beam from the second light source to the second imaging module and to transmit reflected light of the sample arm portion from the second imaging module to the interferometer, the second fiber configured to transmit a reference arm portion of the second light beam from the second light source to the second imaging module, the third fiber being configured to transmit reflected light of the reference arm portion from the second imaging module to the interferometer.

### Embodiment 35

The ultra-wide FOV OCT imaging system in Embodiment 27, further comprising a two-channel optical path difference (OPD) compensation unit disposed in an optical path of the reference arm portion and including a first optical channel, a second optical channel, and an optical switching element, wherein a first optical path of the first optical channel is shorter than a second optical path of the second optical channel, wherein the optical switching element is configured to switch the reference arm portion between the first optical channel and the second optical channel.

### Embodiment 36

The ultra-wide FOV OCT imaging system in Embodiment 27, wherein the second imaging module further includes an optical path difference (OPD) compensator disposed closely to a secondary image plane of the second imaging module within 5 mm, the OPD compensator including a center and a peripheral region, wherein a first optical path of the sample arm portion along the center is shorter than a second optical path of the sample arm portion along the peripheral region.

### Embodiment 37

The ultra-wide FOV OCT imaging system in Embodiment 27, further comprising an optical circulator including three ports and disposed in at least one of an optical path the sample arm or an optical path of the reference arm.

### Embodiment 38

The ultra-wide FOV OCT imaging system in Embodiment 27, wherein a field of view of the OCT imaging system is about 130 degrees by 130 degrees in a single volume acquisition.

### Embodiment 39

An ultra-wide field of view (FOV) optical coherence tomography (OCT) imaging system, comprising:

an optical window disposed at a distal end of an imaging probe and configured to be in contact with a cornea of an eye;

a first imaging module disposed inside the imaging probe and configured to form a first image of the eye, the first imaging module including a first light source and a light conditioning element, the first light source configured to provide a first light beam, the light conditioning element configured to direct the first light beam through the optical window to the eye;

a second imaging module disposed inside the imaging probe and configured to receive a second light beam from a second light source, the second light source being configured to provide the second light beam, the second light beam including a sample arm portion and a reference arm portion;

a beam splitter disposed inside the imaging probe and configured to transmit the first light beam and to reflect the sample arm portion of the second light beam; and

a two-channel optical path difference (OPD) compensation unit disposed in an optical path of the reference arm portion and including a first optical channel, a second optical channel, and an optical switching element, wherein a first optical path of the first optical channel is shorter than a second optical path of the second optical channel, wherein the optical switching element is configured to switch the reference arm portion between the first optical channel and the second optical channel such that an axial optical depth of the OCT imaging system is doubled, thereby a field of view of the OCT imaging system is extended.

### Embodiment 40

The ultra-wide FOV OCT imaging system in Embodiment 39, wherein the optical switching element includes a fast rotating scan mirror.

### Embodiment 41

The ultra-wide FOV OCT imaging system in Embodiment 39, further comprising an aiming light through the first fiber into the second imaging module, wherein the first imaging module is configured to provide a registration for the second imaging module.

### Embodiment 42

The ultra-wide FOV OCT imaging system in Embodiment 39, wherein the first imaging module has a field of view of 130 degrees.

### Embodiment 43

The ultra-wide FOV OCT imaging system in Embodiment 39, wherein the second imaging module further includes a MEM scanning mirror configured to receive the sample arm portion of the second light beam and scan the sample arm portion.

### Embodiment 44

The ultra-wide FOV OCT imaging system in Embodiment 39, further comprising a console and a cable coupled between the console and the imaging probe.

### Embodiment 45

The ultra-wide FOV OCT imaging system in Embodiment 39, wherein the console includes the second light source, an interferometer, and a processor, wherein the cable including a first fiber a second fiber, and a third fiber, the first fiber configured to transmit a sample arm portion of the second light beam from the second light source to the second imaging module and to transmit reflected light of the sample arm portion from the second imaging module to the interferometer, the second fiber configured to transmit a reference arm portion of the second light beam from the second light source to the second imaging module, the third fiber being configured to transmit reflected light of the reference arm portion from the second imaging module to the interferometer.

### Embodiment 46

The ultra-wide FOV OCT imaging system in Embodiment 39, further comprising a dispersion compensation module configured to perform dispersion compensation for the OCT imaging system.

### Embodiment 47

The ultra-wide FOV OCT imaging system in Embodiment 39, wherein the second imaging module further includes an optical path difference (OPD) compensator disposed closely to a secondary image plane of the second imaging module within 5 mm, the OPD compensator including a center and a peripheral region, wherein a first optical path of the sample arm portion along the center is shorter than a second optical path of the sample arm portion along the peripheral region.

### Embodiment 48

The ultra-wide FOV OCT imaging system in Embodiment 39, further comprising an optical circulator including three ports and disposed in at least one of an optical path the sample arm or an optical path of the reference arm.

### Embodiment 49

The ultra-wide FOV OCT imaging system in Embodiment 39, wherein a field of view of the OCT imaging system is 130 degrees by 130 degrees in a single volume acquisition.

### Embodiment 50

An ultra-wide field of view (FOV) optical coherence tomography (OCT) imaging system, comprising:

an optical window disposed at a distal end of an imaging probe and configured to be in contact with a cornea of an eye;

a first imaging module disposed inside the imaging probe and configured to form a first image of the eye, the first imaging module including a first light source and a light conditioning element, the first light source configured to provide a first light beam, the light conditioning element configured to direct the first light beam through the optical window to the eye;

a second imaging module disposed inside the imaging probe and configured to receive a second light beam from a second light source, the second light source being configured to provide the second light beam, the second light beam including a sample arm portion and a reference arm portion;

a beam splitter disposed inside the imaging probe and configured to transmit the first light beam and to reflect the sample arm portion of the second light beam; and

an optical circulator disposed in an optical path of a light beam portion, the light beam portion includes at least one of the sample arm portion or the reference arm portion, the optical circulator including a first port, a second port, and a third port, the first port being configured to receive the light beam portion and to transmit the light beam portion to the second port, the second port being configured to receive retuned light of the light beam portion and to transmit the retuned light of the light beam portion to the third port.

### Embodiment 51

The ultra-wide FOV OCT imaging system in Embodiment 50, wherein the optical circulator is configured for the second light source with wavelength range from 800 nm to 1100 nm.

### Embodiment 52

The ultra-wide FOV OCT imaging system in Embodiment 50, further comprising an aiming light through the first fiber into the second imaging module, wherein the first imaging module is configured to provide a registration for the second imaging module.

### Embodiment 53

The ultra-wide FOV OCT imaging system in Embodiment 50, wherein the first imaging module has a field of view of at least 130 degrees.

### Embodiment 54

The ultra-wide FOV OCT imaging system in Embodiment 50, wherein the second imaging module further includes a MEM scanning mirror configured to receive the sample arm portion of the second light beam and scan the sample arm portion.

### Embodiment 55

The ultra-wide FOV OCT imaging system in Embodiment 50, further comprising a console and a cable coupled between the console and the imaging probe.

### Embodiment 56

The ultra-wide FOV OCT imaging system in Embodiment 55, wherein the console includes the second light source, an interferometer, and a processor, wherein the cable including a first fiber and a second fiber, the first fiber configured to transmit a sample arm portion of the second light beam from the second light source to the second imaging module and to transmit reflected light of the sample arm portion from the second imaging module to the interferometer, the second fiber configured to transmit a reference arm portion of the second light beam from the second light source to the second imaging and to transmit reflected light of the reference arm portion from the second imaging module to the interferometer.

### Embodiment 57

The ultra-wide FOV OCT imaging system in Embodiment 50, further comprising a dispersion compensation module configured to perform dispersion compensation for the OCT imaging system.

### Embodiment 58

The ultra-wide FOV OCT imaging system in Embodiment 50, wherein the second imaging module further includes an optical path difference (OPD) compensator disposed closely to a secondary image plane of the second imaging module within 5 mm, the OPD compensator including a center and a peripheral region, wherein a first optical path of the sample arm portion along the center is shorter than a second optical path of the sample arm portion along the peripheral region.

### Embodiment 59

The ultra-wide FOV OCT imaging system in Embodiment 50, further comprising a two-channel optical path difference (OPD) compensation unit disposed in an optical path of the reference arm portion and including a first optical channel, a second optical channel, and an optical switching element, wherein a first optical path of the first optical channel is shorter than a second optical path of the second optical channel, wherein the optical switching element is configured to switch the reference arm portion between the first optical channel and the second optical channel.

### Embodiment 60

The ultra-wide FOV OCT imaging system in Embodiment 50, wherein a field of view of the OCT imaging system is about 130 degrees by 130 degrees in a single volume acquisition.

While the present disclosure has been disclosed in example embodiments, those of ordinary skill in the art will recognize and appreciate that many additions, deletions and modifications to the disclosed embodiments and their variations may be implemented without departing from the scope of the disclosure. A wide range of variations to those implementations and embodiments described herein are possible. Components and/or features may be added, removed, rearranged, or combinations thereof. Similarly, method steps may be added, removed, and/or reordered.

Likewise various modifications to the implementations described in this disclosure may be readily apparent to those skilled in the art, and the generic principles defined herein may be applied to other implementations without departing from the spirit or scope of this disclosure. Thus, the claims are not intended to be limited to the implementations shown herein, but are to be accorded the widest scope consistent with this disclosure, the principles and the novel features disclosed herein.

Accordingly, reference herein to a singular item includes the possibility that a plurality of the same item may be present. More specifically, as used herein and in the appended claims, the singular forms “a,” “an,” “said,” and “the” include plural referents unless specifically stated otherwise. In other words, use of the articles allow for “at least one” of the subject item in the description above as well as the claims below.

Additionally as used herein, a phrase referring to “at least one of” a list of items refers to any combination of those items, including single members. As an example, “at least one of: a, b, or c” is intended to cover: a, b, c, a-b, a-c, b-c, and a-b-c.

Certain features that are described in this specification in the context of separate embodiments also can be implemented in combination in a single embodiment. Conversely, various features that are described in the context of a single embodiment also can be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations may be described as occurring in a particular order, this should not be understood as requiring that such operations be performed in the particular order described or in sequential order, or that all described operations be performed, to achieve desirable results. Further, other operations that are not disclosed can be incorporated in the processes that are described herein. For example, one or more additional operations can be performed before, after, simultaneously, or between any of the disclosed operations. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single product or packaged into multiple products. Additionally, other embodiments are within the scope of the following claims. In some cases, the actions recited in the claims can be performed in a different order and still achieve desirable results.

The systems, devices, and methods of the preferred embodiments and variations thereof can be embodied and/or implemented at least in part as a machine configured to receive a computer-readable medium storing computer-readable instructions. The instructions are preferably executed by computer-executable components preferably integrated with the system including the computing device configured with software. The computer-readable medium can be stored on any suitable computer-readable media such as RAMs, ROMs, flash memory, EEPROMs, optical devices (e.g., CD or DVD), hard drives, floppy drives, or any suitable device. The computer-executable component is preferably a general or application-specific processor, but any suitable dedicated hardware or hardware/firmware combination can alternatively or additionally execute the instructions.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived therefrom, such that structural and logical substitutions and changes may be made without departing from the scope of this disclosure. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. An ultra-wide field of view (FOV) optical coherence tomography (OCT) imaging system, comprising:
an imaging probe including an optical window, a first imaging module, and a second imaging module, the optical window configured to be in contact with a cornea of an eye, the first imaging module including a first light source and configured to direct a first light beam of the first light source through the optical window to the eye and to form a first image of the eye, the second imaging module configured to receive a second light beam from a second light source and to direct the second light beam through the optical window to the eye and to receive reflected light of the second light beam from the eye;
a console including the second light source, an interferometer configured to receive the reflected light from the second imaging module and to generate data, and a processor configured to process the data from the interferometer and to generate the second image; and
a cable coupled between the console and the imaging probe, the cable including a first fiber and a second fiber, the first fiber configured to transmit a sample arm portion of the second light beam from the second light source to the second imaging module and to transmit reflected light of the sample arm portion from the second imaging module to the interferometer, the second fiber configured to transmit a reference arm portion of the second light beam from the second light source to the second imaging module.

2. The ultra-wide FOV OCT imaging system in claim 1, wherein the cable further includes a third fiber, the third fiber being configured to transmit reflected light of the reference arm portion from the second imaging module to the interferometer.

3. The ultra-wide FOV OCT imaging system in claim 2, wherein the first fiber, the second fiber and the third fiber are closely fixed inside the cable such that external motion effects cause same changes in polarization direction and optical path lengths for the first fiber, the second fiber and the third fiber.

4. The ultra-wide FOV OCT imaging system in claim 2, wherein the first fiber, the second fiber and the third fiber are polarization maintaining fibers.

5. The ultra-wide FOV OCT imaging system in claim 1, wherein the imaging probe further includes a reflection module, the reflection module being configured to receive the reference arm portion of the second light beam from the second light source and to reflect back the reference arm portion to the interferometer.

6. The ultra-wide FOV OCT imaging system in claim 1, further comprising an aiming light through the first fiber into the second imaging module, wherein the first imaging module is configured to provide a registration for the second imaging module.

7. The ultra-wide FOV OCT imaging system in claim 1, wherein the first imaging module further includes a light conditioning element configured to directional control the first light beam to the eye, wherein the first imaging module has a field of view of 130 degrees.

8. The ultra-wide FOV OCT imaging system in claim 1, wherein the second imaging module further includes a MEM scanning mirror configured to receive the sample arm portion of the second light beam and scan the sample arm portion.

9. The ultra-wide FOV OCT imaging system in claim 1, wherein the second imaging module further includes a beam splitter configured to transmit the first light beam and to reflect the sample arm portion of the second light beam.

10. The ultra-wide FOV OCT imaging system in claim 1, wherein the second imaging module further includes an optical path difference (OPD) compensator disposed closely to a secondary image plane of the second imaging module within 1 mm, the OPD compensator including a center and a peripheral region, wherein a first optical path of the sample arm portion along the center is shorter than a second optical path of the sample arm portion along the peripheral region.

11. The ultra-wide FOV OCT imaging system in claim 1, further comprising a two-channel optical path difference (OPD) compensation unit disposed in an optical path of the reference arm portion and including a first optical channel, a second optical channel, and an optical switching element, wherein a first optical path of the first optical channel is shorter than a second optical path of the second optical channel, wherein the optical switching element is configured to switch the reference arm portion between the first optical channel and the second optical channel.

12. The ultra-wide FOV OCT imaging system in claim 1, further comprising a dispersion compensation module disposed in the reference arm portion and configured to perform dispersion compensation for a full field of view of the OCT imaging system such that an axial resolution of the OCT imaging system is improved.

13. The ultra-wide FOV OCT imaging system in claim 1, further comprising an optical circulator including three ports and disposed in at least one of an optical path the sample arm or an optical path of the reference arm.

14. The ultra-wide FOV OCT imaging system in claim 1, wherein a field of view of the OCT imaging system is 130 degrees by 130 degrees in a single volume acquisition.

15. An ultra-wide field of view (FOV) optical coherence tomography (OCT) imaging system, comprising:
an imaging probe including
an optical window configured to be in contact with a cornea of an eye;
a first imaging module configured to form a first image of the eye, the first imaging module including a first light source and a light conditioning element, the first light source configured to provide a first light beam, the light conditioning element configured to direct the first light beam through the optical window to the eye;
a second imaging module configured to form a second image of the eye, the second imaging module including a scanning mirror configured to receive a sample arm portion of a second light beam from a second light source and scan the sample arm portion;
a beam splitter configured to transmit the first light beam and to reflect the sample arm portion of the second light beam; and
an optical path difference (OPD) compensator disposed in an optical path of the sample arm portion, the OPD compensator including a center and a peripheral region, wherein a first optical path of the sample arm portion along the center is shorter than a second optical path of the sample arm portion along the peripheral region such that an axial optical depth of the eye is reduced, thereby a FOV of the OCT imaging system is extended.

16. The ultra-wide FOV OCT imaging system in claim 15, wherein the OPD compensator is disposed closely to a secondary image plane of the second imaging module within 5 mm.

17. The ultra-wide FOV OCT imaging system in claim 15, wherein the optical path difference between the first optical path and the second optical path is between 0.5 mm to 3 mm.

18.-26. (canceled)

27. An ultra-wide field of view (FOV) optical coherence tomography (OCT) imaging system, comprising:
an optical window disposed at a distal end of an imaging probe and configured to be in contact with a cornea of an eye;
a first imaging module disposed inside the imaging probe and configured to form a first image of the eye, the first imaging module including a first light source and a light conditioning element, the first light source configured to provide a first light beam, the light conditioning element configured to direct the first light beam through the optical window to the eye;
a second imaging module disposed inside the imaging probe and configured to receive a second light beam from a second light source, the second imaging module includes a plurality of optical lenses, the second light source being configured to provide the second light beam, the second light beam including a sample arm portion and a reference arm portion;
a beam splitter disposed inside the imaging probe and configured to transmit the first light beam and to reflect the sample arm portion of the second light beam; and
a dispersion compensation module disposed in an optical path of the reference arm portion and configured to perform dispersion compensation of the OCT imaging system;
wherein the plurality of optical lenses includes a plurality of optical materials such that a difference between a first total dispersion of a first optical path length of the sample arm portion in the center and a second total dispersion of a second optical path length of the sample arm portion along the peripheral region of a full FOV is reduced and an axial resolution of a second image is improved.

28. The ultra-wide FOV OCT imaging system in claim 27, wherein the plurality of optical materials are configured to reduce an optical path difference (OPD) between a longest wavelength and a shortest wavelength in a wavelength range of the second light source to be a constant for the full FOV of the OCT imaging system.

29. The ultra-wide FOV OCT imaging system in claim 27, wherein the dispersion compensation module is configured to compensate a residual OPD for the full FOV of the OCT imaging system.

30.-38. (canceled)

39. An ultra-wide field of view (FOV) optical coherence tomography (OCT) imaging system, comprising:
an optical window disposed at a distal end of an imaging probe and configured to be in contact with a cornea of an eye;
a first imaging module disposed inside the imaging probe and configured to form a first image of the eye, the first imaging module including a first light source and a light conditioning element, the first light source configured to provide a first light beam, the light conditioning element configured to direct the first light beam through the optical window to the eye;
a second imaging module disposed inside the imaging probe and configured to receive a second light beam from a second light source, the second light source being configured to provide the second light beam, the second light beam including a sample arm portion and a reference arm portion;
a beam splitter disposed inside the imaging probe and configured to transmit the first light beam and to reflect the sample arm portion of the second light beam; and
a two-channel optical path difference (OPD) compensation unit disposed in an optical path of the reference arm portion and including a first optical channel, a second optical channel, and an optical switching element, wherein a first optical path of the first optical channel is shorter than a second optical path of the second optical channel, wherein the optical switching element is configured to switch the reference arm portion between the first optical channel and the second optical channel such that an axial optical depth of the OCT imaging system is doubled, thereby a field of view of the OCT imaging system is extended.

40. The ultra-wide FOV OCT imaging system in claim 39, wherein the optical switching element includes a fast rotating scan mirror.

41.-49. (canceled)

50. An ultra-wide field of view (FOV) optical coherence tomography (OCT) imaging system, comprising:
an optical window disposed at a distal end of an imaging probe and configured to be in contact with a cornea of an eye;
a first imaging module disposed inside the imaging probe and configured to form a first image of the eye, the first imaging module including a first light source and a light conditioning element, the first light source configured to provide a first light beam, the light conditioning element configured to direct the first light beam through the optical window to the eye;
a second imaging module disposed inside the imaging probe and configured to receive a second light beam from a second light source, the second light source being configured to provide the second light beam, the second light beam including a sample arm portion and a reference arm portion;
a beam splitter disposed inside the imaging probe and configured to transmit the first light beam and to reflect the sample arm portion of the second light beam; and
an optical circulator disposed in an optical path of a light beam portion, the light beam portion includes at least one of the sample arm portion or the reference arm portion, the optical circulator including a first port, a second port, and a third port, the first port being configured to receive the light beam portion and to transmit the light beam portion to the second port, the second port being configured to receive retuned light of the light beam portion and to transmit the retuned light of the light beam portion to the third port.

51. The ultra-wide FOV OCT imaging system in claim 50, wherein the optical circulator is configured for the second light source with wavelength range from 800 nm to 1100 nm.

52.-60. (canceled)
